(19) United States
(12) Patent Application Publication
(10) Pub. No.: US 2026/0028645 A1
(43) Pub. Date: Jan. 29, 2026

(54) METHODS AND MATERIALS FOR PROMOTING BONE GROWTH

(71) Applicant: Mayo Foundation for Medical Education and Research, Rochester, MN (US)

(72) Inventors: Erin B. McGlinch, Rochester, MN (US); Joseph A. Panos, Rochester, MN (US); Michael A. Barry, Rochester, MN (US); Christopher H. Evans, Cohasset, MA (US)

(21) Appl. No.: 19/264,151

(22) Filed: Jul. 9, 2025

Related U.S. Application Data
(60) Provisional application No. 63/669,520, filed on Jul. 10, 2024.

(51) Int. Cl. C12N 15/86; A61K 38/17; A61K 38/18; A61K 48/00; A61P 19/00; C07K 14/51; C07K 14/715

(52) U.S. Cl. CPC C12N 15/86; A61K 38/1793; A61K 38/1875; A61K 48/005; A61P 19/00; C07K 14/51; C07K 14/7155; C12N 2710/10043; C12N 2750/14143

(57) ABSTRACT

This document provides methods and materials involved in promoting bone growth. For example, this document provides vectors designed to express (a) a nucleotide sequence encoding a bone morphogenetic protein 2 (BMP2) polypeptide and/or (b) a nucleotide sequence encoding an interleukin-1 receptor antagonist (IL-1Ra) polypeptide for promoting bone growth. In some cases, one or more vectors provided herein can be administered to a mammal (e.g., a human) having a disease, disorder, or condition associated with bone loss to treat the mammal. For example, a population of a single vector provided herein can be used to increase expression of a BMP2 polypeptide and an IL-1Ra polypeptide by cells within a mammal (e.g., a human) having a disease, disorder, or condition associated with bone loss to promote bone growth within the mammal.

### CLAIM OF PRIORITY

This application claims the benefit of U.S. Provisional Patent Application Ser. No. 63/669,520, filed on Jul. 10, 2024. The entire contents of which are hereby incorporated by reference.

### STATEMENT REGARDING FEDERAL FUNDING

This invention was made with government support under AR056950 and AR079842 awarded by the National Institutes of Health. The government has certain rights in the invention.

### SEQUENCE LISTING

This application contains a Sequence Listing that has been submitted electronically as an XML file named “07039-2341001_SL_ST26.XML.” The XML file, created on Oct. 9, 2025, is 7,977 bytes in size. The material in the XML file is hereby incorporated by reference in its entirety.

### TECHNICAL FIELD

This document relates to methods and materials involved in promoting bone growth and/or bone regeneration. For example, this document provides vectors (e.g., viral vectors and non-viral vectors) designed to express (a) a nucleotide sequence encoding a bone morphogenetic protein (BMP) polypeptide (e.g., a BMP2 polypeptide) and/or (b) a nucleotide sequence encoding a polypeptide that can inhibit an interleukin-1 (IL-1) polypeptide (e.g., an interleukin-1 receptor antagonist (IL-1Ra) polypeptide). In some cases, a viral vector having a genome (e.g., a single-stranded or double-stranded DNA genome) including (a) a nucleotide sequence encoding a BMP polypeptide and/or (b) a nucleotide sequence encoding a polypeptide that can inhibit an IL-1 polypeptide can be used for promoting bone growth and/or bone regeneration. In some cases, a viral vector designed to express (a) a nucleotide sequence encoding a BMP polypeptide and/or (b) a nucleotide sequence encoding a polypeptide that can inhibit an IL-1 polypeptide can increase expression of a BMP polypeptide and/or a polypeptide that can inhibit an IL-1 polypeptide on or within a cell (e.g., on or within a cell within a mammal such as a human). In some cases, one or more vectors provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal (e.g., a human) having a disease, disorder, or condition associated with bone loss to treat the mammal. For example, a population of a single viral vector provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be used to increase expression of a BMP polypeptide and/or a polypeptide that can inhibit an IL-1 polypeptide by cells within a mammal (e.g., a human) having a disease, disorder, or condition associated with bone loss to promote bone growth and/or bone regeneration within the mammal.

### BACKGROUND

Nearly 25 million individuals will experience a traumatic musculoskeletal injury each year resulting in $170 billion in healthcare-associated costs (De la Vega et al., Transl. Res., 236:1-16 (2021); and Zura et al., JAMA Surg., 151: e162775 (2016)). Skeletal fractures comprise more than half of all traumatic injuries and represent a significant source of disability in young, otherwise healthy adults. Although the long bones of the skeleton are one of the few human organs that can regenerate scarlessly after injury, approximately 100,000 patients each year in the United States will suffer severe disability from bone defects that fail to heal (Zura et al., JAMA Surg., 151: e162775 (2016)). Management of fracture non-union constitutes a major clinical burden and accumulates $9.2 billion per year in direct medical costs (Non-union fractures market: global industry trend analysis 2013 to 2017 and forecast 2018-2026, persistencemarketresearch.com/market-research/non-union-fractures-market.asp (2020)). Large segmental defects are a particular problem because, beyond a certain critical size, they fail to heal even in young, healthy individuals (Huang et al., Novel Techniques and Future Perspective for Investigating Critical-Size Bone Defects, Bioengineering (Basel), 9 (2022); and Schultz et al., Bull. Hosp. Jt. Dis., 80:53-64 (2022)).

Autologous bone graft is the treatment of choice for non-unions, but has the disadvantages of limited supply, the need for second site surgery, and donor site morbidity (Azi et al., BMC Musculoskelet. Disord., 17:465 (2016)). Allograft, in contrast, is available in almost unlimited amounts as an off-the-shelf product. However, the processing involved in producing allograft kills bone cells, leaving dead bone that can serve as a space filler but lacks osteoinductive properties and does not remodel (De la Vega et al., Transl. Res., 236:1-16 (2021); and Schultz et al., Bull. Hosp. Jt. Dis., 80:53-64 (2022)). Further, allograft does not integrate into host bone and cannot repair microdamage, so over 40% of structural allografts fail within 5 years (Gharedaghi et al., Arch. Bone Jt. Surg., 4:236-242 (2016)). There also remain lingering concerns about disease transmission when using allograft bone. The clinical need for reliable osteogenic materials is reflected in data showing that approximately 1.6 million autograft procedures are conducted each year in the USA and bone is the second most frequently allografted tissue after blood (Baldwin et al., J. Orthop. Trauma, 33:203-213 (2019)).

Large segmental defects can also be healed surgically by the Ilizarov technique, otherwise known as distraction osteogenesis (Gubin et al., Bull. Hosp. Jt. Dis., 74:145-154 (2016); and Zhang et al., J. Orthop. Surg. Res., 12:183 (2017)). This method is based upon the ability to stimulate bone growth by slowly pulling apart two cut ends of bone. The surgery is complex and a cumbersome, circular external fixator is used to stabilize the defect. Because new bone is formed at a rate of only about 1 mm/day, it takes considerable time to fill in large defects and many patients find the daily distraction procedure painful. Furthermore, it cannot be applied along the entire length of a long bone because the ends do not permit placing of the Ilizarov pins. Pin tract infections occur often and need immediate treatment in order to prevent progression to osteomyelitis.

### SUMMARY

This document provides methods and materials involved in promoting bone growth and/or bone regeneration. For example, this document provides vectors (e.g., viral vectors and non-viral vectors) designed to express (a) a nucleotide sequence encoding a BMP polypeptide (e.g., a BMP2 polypeptide) and/or (b) a nucleotide sequence encoding a polypeptide that can inhibit an IL-1 polypeptide (e.g., an IL-1Ra polypeptide). In some cases, a viral vector having a genome (e.g., a single-stranded or double-stranded DNA genome) including a nucleic acid sequence including (a) a nucleotide sequence encoding a BMP polypeptide and/or (b) a nucleotide sequence encoding a polypeptide that can inhibit an IL-1 polypeptide can be used for promoting bone growth and/or bone regeneration. In some cases, vectors provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can increase expression of a BMP polypeptide and/or a polypeptide that can inhibit an IL-1 polypeptide on or within a cell (e.g., on or within a cell within a mammal such as a human). In some cases, one or more vectors provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal (e.g., a human) having a disease, disorder, or condition associated with bone loss to treat the mammal. For example, a population of a single viral vector provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be used to increase expression of a BMP polypeptide and/or a polypeptide that can inhibit an IL-1 polypeptide by cells within a mammal (e.g., a human) having a disease, disorder, or condition associated with bone loss to promote bone growth and/or bone regeneration within the mammal.

As demonstrated herein, expression of a BMP2 polypeptide and an IL-1Ra polypeptide driven by a single viral vector can reduce bone loss or increase bone growth and/or bone regeneration within a mammal (e.g., without inducing a significant adverse inflammatory immune response within the mammal). Having the ability to promote bone growth and/or bone regeneration as described herein (e.g., by administering one or more vectors provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide)) provides a unique and unrealized opportunity to treat mammals (e.g., humans) having a disease, disorder, or condition associated with bone loss without inducing a significant adverse inflammatory immune response within the mammal.

In general, one aspect of this document features vectors that include a promotor sequence operably linked to a nucleic acid comprising (a) a nucleotide sequence encoding a bone morphogenetic protein (BMP) polypeptide and (b) a nucleotide sequence encoding a polypeptide that can inhibit an interleukin-1 (IL-1) polypeptide. The vector can be a viral vector (e.g., an adeno-associated viral vector or a helper-dependent adenoviral vector). The promoter can be a CMV promoter, a EF1-α promoter, a SV40 promoter, a Cbh promoter, a T7 promoter, a RSV promoter, a PGK promoter, a CAG promoter, or a β-actin promoter. (a) and (b) can be separated by a nucleic acid sequence encoding a P2A polypeptide. (a) and (b) can be separated by a polypeptide cleavage site (e.g., a furin cleavage site). (a) and (b) can be separated by an internal ribosome entry site. (a) and (b) can be separated by a second promoter. The second promoter can be a CMV promoter, a EF1-α promoter, a SV40 promoter, a Cbh promoter, a T7 promoter, a RSV promoter, a PGK promoter, a CAG promoter, or a β-actin promoter. The BMP polypeptide can be a BMP2 polypeptide. The BMP polypeptide can have the amino acid sequence set forth in SEQ ID NO:1. The polypeptide that can inhibit said IL-1 polypeptide can be an IL-1Ra polypeptide. The polypeptide that can inhibit said IL-1 polypeptide can have the amino acid sequence set forth in SEQ ID NO:3.

In another aspect, this document features methods for treating a mammal having a disease, disorder, or condition associated with bone loss. The methods can include, or consist essentially of, administering one or more vectors that include a promotor sequence operably linked to a nucleic acid comprising (a) a nucleotide sequence encoding a bone morphogenetic protein (BMP) polypeptide and (b) a nucleotide sequence encoding a polypeptide that can inhibit an interleukin-1 (IL-1) polypeptide to a mammal having a disease, disorder, or condition associated with bone loss, wherein expression of said BMP polypeptide and said polypeptide that can inhibit said IL-1 polypeptide is increased within said mammal, and wherein bone loss is reduced within said mammal or bone growth is increased within said mammal. The mammal can be a human. The disease, disorder, or condition can be a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, or a cartilage grafting procedure. The administration can include direct injection in situ, intravenous injection, intra-arterial injection, or intramuscular injection. The administration can result in expression of less than 12 mg of said BMP polypeptide. The administration can result in expression of from about 1.2 mg of said BMP polypeptide to about 12 mg of said BMP polypeptide.

In another aspect, this document features methods for promoting bone growth within a mammal. The methods can include, or consist essentially of, administering one or more vectors that include a promotor sequence operably linked to a nucleic acid comprising (a) a nucleotide sequence encoding a bone morphogenetic protein (BMP) polypeptide and (b) a nucleotide sequence encoding a polypeptide that can inhibit an interleukin-1 (IL-1) polypeptide to a mammal, wherein expression of said BMP polypeptide and said polypeptide that can inhibit said IL-1 polypeptide is increased within said mammal, and wherein bone growth is increased within said mammal. The mammal can be a human. The disease, disorder, or condition can be a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, or a cartilage grafting procedure. The administration can include direct injection in situ, intravenous injection, intra-arterial injection, or intramuscular injection. The administration can result in expression of less than 12 mg of said BMP polypeptide. The administration can result in expression of from about 1.2 mg of said BMP polypeptide to about 12 mg of said BMP polypeptide.

In another aspect, this document features compositions including (1) a first vector comprising nucleic acid comprising a first promotor sequence operably linked to a nucleotide sequence encoding a bone morphogenetic protein (BMP) polypeptide and (2) a second vector comprising a second promotor sequence operably linked to a nucleotide sequence encoding a polypeptide that can inhibit an interleukin-1 (IL-1) polypeptide. The first vector and said second vector can be viral vectors. The first vector and said second vector can independently be an adeno-associated viral vector or a helper-dependent adenoviral vector. The first promoter can be a CMV promoter, a EF1-α promoter, a SV40 promoter, a Cbh promoter, a T7 promoter, a RSV promoter, a PGK promoter, a CAG promoter, or a β-actin promoter. The second promoter can be a CMV promoter, a EF1-α promoter, a SV40 promoter, a Cbh promoter, a T7 promoter, a RSV promoter, a PGK promoter, a CAG promoter, or a β-actin promoter. The BMP polypeptide can be a BMP2 polypeptide. The BMP polypeptide can have the amino acid sequence set forth in SEQ ID NO:1. The polypeptide that can inhibit said IL-1 polypeptide can be a IL-1Ra polypeptide. The polypeptide that can inhibit said IL-1 polypeptide can have the amino acid sequence set forth in SEQ ID NO:3.

In another aspect, this document features methods for treating a mammal having a disease, disorder, or condition associated with bone loss. The methods can include, or consist essentially of, administering a composition including (1) a first vector comprising nucleic acid comprising a first promotor sequence operably linked to a nucleotide sequence encoding a bone morphogenetic protein (BMP) polypeptide and (2) a second vector comprising a second promotor sequence operably linked to a nucleotide sequence encoding a polypeptide that can inhibit an interleukin-1 (IL-1) polypeptide to a mammal having a disease, disorder, or condition associated with bone loss, wherein expression of said BMP polypeptide and said polypeptide that can inhibit said IL-1 polypeptide is increased within said mammal, and wherein bone loss is reduced within said mammal or bone growth is increased within said mammal. The mammal can be a human. The disease, disorder, or condition can be a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, or a cartilage grafting procedure. The administration can include direct injection in situ, intravenous injection, intra-arterial injection, or intramuscular injection. The administration can result in expression of less than 12 mg of said BMP polypeptide. The administration can result in expression of from about 1.2 mg of said BMP polypeptide to about 12 mg of said BMP polypeptide.

In another aspect, this document features methods for promoting bone growth within a mammal. The methods can include, or consist essentially of, administering a composition including (1) a first vector comprising nucleic acid comprising a first promotor sequence operably linked to a nucleotide sequence encoding a bone morphogenetic protein (BMP) polypeptide and (2) a second vector comprising a second promotor sequence operably linked to a nucleotide sequence encoding a polypeptide that can inhibit an interleukin-1 (IL-1) polypeptide to a mammal, wherein expression of said BMP polypeptide and said polypeptide that can inhibit said IL-1 polypeptide is increased within said mammal, and wherein bone growth is increased within said mammal. The mammal can be a human. The disease, disorder, or condition can be a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, and a cartilage grafting procedure. The administration can include direct injection in situ, intravenous injection, intra-arterial injection, or intramuscular injection. The administration can result in expression of less than 12 mg of said BMP polypeptide. The administration can result in expression of from about 1.2 mg of said BMP polypeptide to about 12 mg of said BMP polypeptide.

In another aspect, this document features methods for treating a mammal having a disease, disorder, or condition associated with bone loss. The methods can include, or consist essentially of, administering, to a mammal having a disease, disorder, or condition associated with bone loss, (a) a first vector comprising nucleic acid comprising a first promotor sequence operably linked to a nucleotide sequence encoding a bone morphogenetic protein (BMP) polypeptide and (b) a second vector comprising a second promotor sequence operably linked to a nucleotide sequence encoding a polypeptide that can inhibit an interleukin-1 (IL-1) polypeptide, wherein expression of said BMP polypeptide and said polypeptide that can inhibit said IL-1 polypeptide is increased within said mammal, and wherein bone loss is reduced within said mammal or bone growth is increased within said mammal. The mammal can be a human. The disease, disorder, or condition can be a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, or a cartilage grafting procedure. The administration can include direct injection in situ, intravenous injection, intra-arterial injection, or intramuscular injection. The administration can result in expression of less than 12 mg of said BMP polypeptide. The administration can result in expression of from about 1.2 mg of said BMP polypeptide to about 12 mg of said BMP polypeptide.

In another aspect, this document features methods for promoting bone growth within a mammal. The methods can include, or consist essentially of, administering, to a mammal, (a) a first vector comprising nucleic acid comprising a first promotor sequence operably linked to a nucleotide sequence encoding a bone morphogenetic protein (BMP) polypeptide and (b) a second vector comprising a second promotor sequence operably linked to a nucleotide sequence encoding a polypeptide that can inhibit an interleukin-1 (IL-1) polypeptide, wherein expression of said BMP polypeptide and said polypeptide that can inhibit said IL-1 polypeptide is increased within said mammal, and wherein bone growth is increased within said mammal. The mammal can be a human. The disease, disorder, or condition can be a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, or a cartilage grafting procedure. The administration can include direct injection in situ, intravenous injection, intra-arterial injection, or intramuscular injection. The administration can result in expression of less than 12 mg of said BMP polypeptide. The administration can result in expression of from about 1.2 mg of said BMP polypeptide to about 12 mg of said BMP polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. A schematic showing an intersection of IL-1 and BMP2 in bone healing. High doses of BMP2 are inflammatory and form poor quality bone via intramembranous ossification. Blocking IL-1 with IL-1Ra can potentiate the activity of BMP2, allowing low concentrations to form high quality bone endochondrally.

FIGS. 2A-2B. Schematics of exemplary viral vectors including (a) a nucleotide sequence encoding a BMP2 polypeptide and/or (b) a nucleotide sequence encoding an IL-1Ra polypeptide. FIG. 2A) Schematics of viral genomic structure of an exemplary AAV (top), a first-generation replication defective (RD) adenovirus (middle), and helper dependent (HD) adenovirus (bottom) are shown, with the positions of exemplary transgenes indicated. FIG. 2B) Schematics of exemplary nucleic acids including (a) a nucleotide sequence encoding a BMP2 polypeptide and/or (b) a nucleotide sequence encoding an IL-1Ra polypeptide. Constructs were designed to encode a BMP2 polypeptide, both a BMP2 polypeptide and an IL-1Ra polypeptide, an IL-1Ra polypeptide alone (IL1RN), or a GFP-luciferase (GL) polypeptide.

FIG. 3. Transduction of monolayer cultures of rat MSCs by different AAV serotypes. A low dose AAV control (AAV.GL) was used at a MOI of 10,000. Images were photographed 48 hours post-transduction.

FIGS. 4A-4B. In vivo induction of IL-1 in a rat segmental defect by adenovirus was comparable to that induced by 11 ug recombinant human BMP (rhBMP). FIG. 4A). IL-1α polypeptide levels 5 days post-procedure. FIG. 4B) IL-1ß polypeptide levels 5 days post-procedure.

FIGS. 5A-5B. Luciferase expression in vivo. 5×10¹⁰ viral genomes (vg) AAV2.5GL were administered to femoral osseous defects in rats. FIG. 5A) Quantified luciferase expression 7 days post-injection. FIG. 5B) In vivo image of luciferase expression 7 days post-injection.

FIG. 6. X-ray images of rat femora 10 days, 8 weeks and 12 weeks after surgical creation of 5 mm, critical size segmental defects. Doses of 1×10¹⁰ vg AAV2.5BMP-2 or AAV2.5BMP-2-IL1RN were administered to the defects on a collagen sponge. AAV2.5BMP-2 alone did not induce new bone formation. AAV2.5BMP-2-IL1RN, in contrast, led to a substantial deposition of new bone within the defect.

FIGS. 7A-7H. Effects of rhBMP2, AAV6-BMP2, and AAV6-BMP2-P2A-IL1Ra on bone regeneration. FIG. 7A) A schematic illustrating the experimental design using a rat critical-sized defect model with a 5 mm segmental femoral defect. Doses of 1×10¹⁰ vg AAV6-BMP2 or 1×10⁹ vg, 2.5×10⁹ vg, 5×10⁹ vg, or 1×10¹⁰ vg AAV6-BMP2-P2A-IL1Ra were administered with a collagen sponge. FIG. 7B) X-ray benchmarks of the 5 mm critical-sized defect model at the day 10, week 4, week 8, and week 10 timepoints are shown. An empty control was used as a negative control to demonstrate the effects of no treatment on the model. The 1 mm osteotomy represents natural bone healing process. The 5 mm treatment with rhBMP2 represents the current clinical standard against which improvements were sought. FIG. 7C) X-ray images of rat femora treated with a low dose of 1×10⁹ vg and the highest dose of 1×10¹⁰ vg AAV6-BMP2-P2A-IL1Ra at day 10, week 4, week 8, week 12, and week 16 are shown. FIG. 7D) X-ray images of rat femora treated with AAV6-BMP2 or AAV6-BMP2-P2A-IL1Ra at day 10, week 4, week 8, week 12, and week 16, demonstrating the contribution of IL1Ra to bone regeneration. FIG. 7E) Ex vivo X-ray images of rat femora treated with increasing doses of AAV6-BMP2-P2A-IL1Ra ranging from 1×10⁹ vg to 1×10¹⁰ vg at week 16. FIG. 7F) Ex vivo X-ray images of rat femora treated with 1×10¹⁰ vg AAV6-BMP2 and AAV6-BMP2-P2A-IL1Ra at week 16, demonstrating the effects of IL1Ra on bone regeneration. FIG. 7G) X-ray and microCT images of rhBMP2 protein therapy (11 μg) at weeks 4 and 8, compared to x-ray and microCT images at week 16 after 1×10¹⁰ vg AAV6-BMP2-P2A-IL1Ra treatment, demonstrating increased quality of bone with the vector therapy. FIG. 7H) Images of dissected femora at week 16 showed that unlike the rat femora treated with rhBMP2 and AAV6-BMP2, treatment with multiple doses of AAV6-BMP2-P2A-IL1Ra resulted in cartilage-appearing disks, demonstrating that bone formation had likely proceeded endochondrally.

FIGS. 8A-8G. AAV6-BMP2-P2A-IL1Ra demonstrated that AAV therapy most effectively replicates the natural bone healing process. FIG. 8A. X-ray, histology, and microCT images of intramembranous ossification compared to endochondral ossification. FIG. 8B) Safranin-O staining images of natural healing process of a 1 mm defect (endochondral ossification) and an empty 5 mm defect at 1.25× and 10× magnifications. FIG. 8C) Safranin-O staining images of a 5 mm defect treated with rhBMP2 depicting intramembranous ossification at 1.25× and 10× magnifications. FIG. 8D) X-ray and safranin-O staining images of ex vivo femora treated with 1×10⁹ vg AAV6-BMP2-P2A-IL1Ra at week 16. FIG. 8E) X-ray and safranin-O staining images of ex vivo femora treated with 1×10¹⁰ vg AAV6-BMP2-P2A-IL1Ra at week 16. The defect sites were magnified in both FIGS. 8D and 8E. The presence of chondrocytes indicates endochondral ossification as the bone regeneration pathway, suggesting a natural bone formation pathway with higher quality and more biomechanically stable bone. FIG. 8F) X-ray and safranin-O staining images of ex vivo femora treated with 1×10¹⁰ vg AAV6-BMP2 at week 16. The defect site has been magnified. Failure to bridge the defect is highlighted by the capped defect ends, as shown in the histology. FIG. 8G) X-ray and safranin-O staining images comparing alternative therapies, rhBMP2 and BMP-2 chemically modified RNA (cmRNA), of varying doses at weeks 4 and 8 with 1×10¹⁰ vg AAV6-BMP2-P2A-IL1Ra at week 16 demonstrated that AAV therapy is closest to natural healing.

FIGS. 9A-9D. AAV6-BMP2-P2A-IL1Ra promoted bone regeneration via endochondral ossification. FIG. 9A) MicroCT images of ex vivo femur treated with 1×10¹⁰ vg AAV6-BMP2-P2A-IL1Ra at week 16 depicting the architecture of regenerated bone. FIG. 9B) X-ray and safranin-O staining images showed that 1×10¹⁰ vg of both AAV6-BMP2 and AAV6-BMP2-P2A-IL1Ra increased bone regeneration, with the AAV6-BMP2-P2A-IL1Ra therapy promoting the regeneration via endochondral ossification. FIG. 9C) X-ray, safranin-O staining, and micro-CT images showed that dose escalation from 1×10⁹ vg to 1×10¹⁰ vg AAV6-BMP2-P2A-IL1Ra stimulated bone regeneration via endochondral ossification, with 1×10¹⁰ vg AAV6-BMP2-P2A-IL1Ra showing bone closer to full thickness by week 16. FIG. 9D) X-ray, safranin-O staining, and microCT images showed that treatment with 11 ug rhBMP-2 used intramembranous ossification whereas treatment with 1×10¹⁰ vg AAV6-BMP2-P2A-IL1Ra used endochondral ossification leading to higher quality and more biomechanically stable bone.

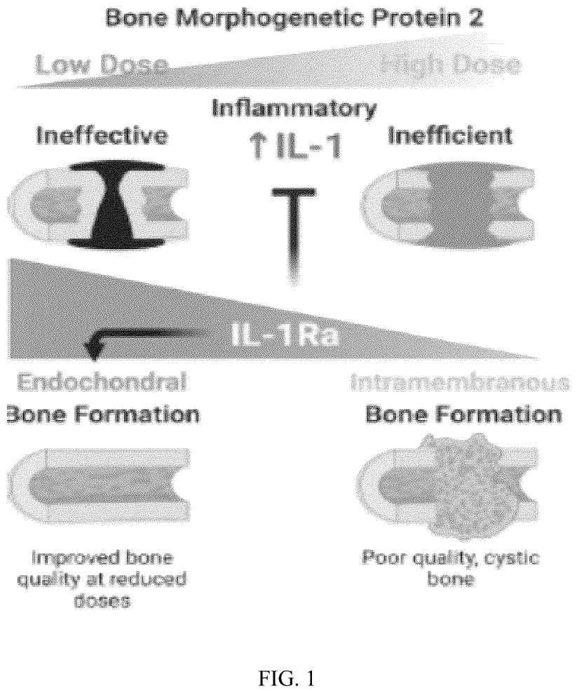

### DETAILED DESCRIPTION

This document provides methods and materials involved in promoting bone growth and/or bone regeneration. For example, this document provides vectors (e.g., viral vectors and non-viral vectors) designed to express (a) a nucleotide sequence encoding a BMP polypeptide (e.g., a BMP2 polypeptide) and/or (b) a nucleotide sequence encoding a polypeptide that can inhibit an IL-1 polypeptide (e.g., an IL-1Ra polypeptide) for promoting bone growth and/or bone regeneration. In some cases, a viral vector provided herein can have a genome (e.g., a single-stranded or double-stranded DNA genome) including a nucleic acid sequence including (a) a nucleotide sequence encoding a BMP polypeptide and/or (b) a nucleotide sequence encoding a polypeptide that can inhibit an IL-1 polypeptide. In some cases, vectors provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can increase expression of a BMP polypeptide and/or a polypeptide that can inhibit an IL-1 polypeptide on or within a cell (e.g., on or within a cell within a mammal such as a human). In some cases, one or more vectors provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal (e.g., a human) having a disease, disorder, or condition associated with bone loss to treat the mammal. For example, a population of a single viral vector provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be used to increase expression of a BMP polypeptide and/or a polypeptide that can inhibit an IL-1 polypeptide by cells within a mammal (e.g., a human) having a disease, disorder, or condition associated with bone loss to promote bone growth and/or bone regeneration within the mammal.

In some cases, the methods and materials provided herein can be used to promote bone formation (e.g., via endochondral ossification), resulting in higher quality bone formation. For example, the methods and materials provided herein can promote bone growth and/or bone regeneration within a mammal (e.g., a human) without the need for repeated dosing of either a BMP polypeptide (e.g., a BMP2 polypeptide) or a polypeptide that can inhibit an IL-1 polypeptide (e.g., an IL-1Ra polypeptide).

In some cases, the methods and materials provided herein can be used to promote bone growth and/or bone regeneration within a mammal (e.g., a human) using a low dose of a BMP polypeptide (e.g., a BMP2 polypeptide). For example, administering a single viral vector that includes (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) to a mammal (e.g., a human) can result in expression of less than 12 mg of the BMP polypeptide. In another example, administering a single viral vector that includes (a) a nucleotide sequence encoding a BMP polypeptide and (b) a nucleotide sequence encoding a polypeptide that can inhibit an IL-1 polypeptide (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) to a mammal (e.g., a human) can result in expression of from about 1.2 mg of the BMP polypeptide to about 12 mg of the BMP polypeptide.

In some cases, the methods and materials provided herein can be used to promote bone growth and/or bone regeneration within a mammal (e.g., a human) without inducing a significant adverse inflammatory immune response within the mammal. For example, administering a single viral vector that includes (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) to a mammal (e.g., a human) can increase expression of the BMP polypeptide and the IL-1Ra polypeptide within the mammal without increasing expression and/or activity of an IL-1 polypeptide.

Any appropriate vector can be designed to express (a) a nucleotide sequence encoding a BMP polypeptide (e.g., a BMP2 polypeptide) and/or (b) a nucleotide sequence encoding a polypeptide that can inhibit an IL-1 polypeptide (e.g., an IL-1Ra polypeptide) (e.g., can be designed to have a genome including a nucleic acid sequence including (a) a nucleotide sequence encoding a BMP polypeptide and (b) a nucleotide sequence encoding a polypeptide that can inhibit an IL-1 polypeptide). In some cases, a vector can be a viral vector. For example, a viral vector designed to express (a) a nucleotide sequence encoding a BMP polypeptide and/or (b) a nucleotide sequence encoding a polypeptide that can inhibit an IL-1 polypeptide can have a single-stranded or double-stranded genome (e.g., a DNA genome or a RNA genome). When containing a single-stranded genome, the viral vector designed to express (a) a nucleotide sequence encoding a BMP polypeptide and/or (b) a nucleotide sequence encoding a polypeptide that can inhibit an IL-1 polypeptide can be a positive-strand virus or a negative-strand virus. In some cases, a viral vector designed to express (a) a nucleotide sequence encoding a BMP polypeptide and/or (b) a nucleotide sequence encoding a polypeptide that can inhibit an IL-1 polypeptide can infect dividing cells. In some cases, a viral vector designed to express (a) a nucleotide sequence encoding a BMP polypeptide and/or (b) a nucleotide sequence encoding a polypeptide that can inhibit an IL-1 polypeptide can infect non-dividing cells. Examples of viral vectors that can be designed to express (a) a nucleotide sequence encoding a BMP polypeptide and/or (b) a nucleotide sequence encoding a polypeptide that can inhibit an IL-1 polypeptide as described herein (e.g., designed to have a genome including a nucleic acid sequence including (a) a nucleotide sequence encoding a BMP polypeptide and (b) a nucleotide sequence encoding a polypeptide that can inhibit an IL-1 polypeptide) include, without limitation, helper-dependent adenoviral vectors, adenoviral vectors (e.g., replication deficient adenoviral vectors), and adeno-associated viral (AAV) vectors (e.g., serotype 2 AAV (AAV2) vectors, AAV2.5 vectors, AAV6 vectors, AAV1 vectors, and AAV8 vectors). In some cases, a vector can be a non-viral vector. Examples of non-viral vectors that can be designed to express (a) a nucleotide sequence encoding a BMP polypeptide and/or (b) a nucleotide sequence encoding a polypeptide that can inhibit an IL-1 polypeptide include, without limitation, plasmids (e.g., expression plasmids).

A vector (e.g., a viral vector or a non-viral vector) provided herein (e.g., a viral vector designed to express (a) a nucleotide sequence encoding a BMP polypeptide (e.g., a BMP2 polypeptide) and/or (b) a nucleotide sequence encoding a polypeptide that can inhibit an IL-1 polypeptide (e.g., an IL-1Ra polypeptide)) can include any appropriate nucleic acid encoding a BMP polypeptide. For example, a vector having a genome (e.g., a single-stranded or double-stranded DNA genome) including a nucleic acid sequence (e.g., an engineered DNA sequence) including (a) a nucleotide sequence encoding a BMP polypeptide and/or (b) a nucleotide sequence encoding a polypeptide that can inhibit an IL-1 polypeptide can include any appropriate nucleic acid encoding a BMP polypeptide.

A nucleic acid encoding a BMP polypeptide (e.g., a BMP2 polypeptide) in a vector (e.g., a viral vector or a non-viral vector) provided herein (e.g., a viral vector designed to express (a) a nucleotide sequence encoding a BMP polypeptide and/or (b) a nucleotide sequence encoding a polypeptide that can inhibit an IL-1 polypeptide (e.g., an IL-1Ra polypeptide)) can encode any appropriate BMP polypeptide. For example, a viral vector having a genome (e.g., a single-stranded or double-stranded DNA genome) including a nucleic acid sequence (e.g., an engineered DNA sequence) including (a) a nucleotide sequence encoding a BMP polypeptide and/or (b) a nucleotide sequence encoding a polypeptide that can inhibit an IL-1 polypeptide can include nucleic acid encoding any appropriate BMP polypeptide. In some cases, a BMP polypeptide can be an osteogenic BMP polypeptide. Examples of BMP polypeptides that can be encoded by a vector provided herein include, without limitation, BMP2 polypeptides, BMP7 polypeptides, and BMP9 polypeptides. In some cases, a BMP polypeptide that can be encoded by a nucleic acid encoding a BMP polypeptide in a vector provided herein can have the amino acid sequence set forth in the National Center for Biotechnology Information (NCBI) databases at Accession Nos. NM_001200.4, NM_017178.2, and NM_007553.3. In some cases, a BMP polypeptide that can be encoded by a nucleic acid encoding a BMP polypeptide in a vector provided herein can have the amino acid sequence set forth in SEQ ID NO:1. In some cases, a BMP polypeptide that can be encoded by a nucleic acid encoding a BMP polypeptide in a vector provided herein can comprise, consist essentially of, or consist of the amino acid sequence set forth in SEQ ID NO:1. In some cases, a BMP polypeptide that can be encoded by a nucleic acid encoding a BMP polypeptide in a vector provided herein can comprise, consist essentially of, or consist of the amino acid sequence set forth in SEQ ID NO:1 with one, two, three, four, five, six, seven, eight, nine, or ten amino acid deletions, additions, substitutions, or combinations thereof. In some cases, a BMP polypeptide that can be encoded by a nucleic acid encoding a BMP polypeptide in a vector provided herein can comprise, consist essentially of, or consist of the amino acid sequence set forth in SEQ ID NO:1 with two or less, three or less, four or less, five or less, six or less, seven or less, eight or less, nine or less, or ten or less amino acid deletions, additions, substitutions, or combinations thereof.

A vector (e.g., a viral vector or a non-viral vector) provided herein (e.g., a viral vector designed to express (a) a nucleotide sequence encoding a BMP polypeptide (e.g., a BMP2 polypeptide) and/or (b) a nucleotide sequence encoding a polypeptide that can inhibit an IL-1 polypeptide (e.g., an IL-1Ra polypeptide)) can include any appropriate nucleic acid encoding a polypeptide that can inhibit an IL-1 polypeptide. For example, a viral vector having a genome (e.g., a single-stranded or double-stranded DNA genome) including a nucleic acid sequence (e.g., an engineered DNA sequence) including (a) a nucleotide sequence encoding a BMP polypeptide and/or (b) a nucleotide sequence encoding a polypeptide that can inhibit an IL-1 polypeptide can include any appropriate nucleic acid encoding a polypeptide that can inhibit an IL-1 polypeptide.

A nucleic acid encoding a polypeptide that can inhibit an IL-1 polypeptide in a vector (e.g., a viral vector or a non-viral vector) provided herein (e.g., a viral vector designed to express (a) a nucleotide sequence encoding a BMP polypeptide (e.g., a BMP2 polypeptide) and/or (b) a nucleotide sequence encoding an IL-1Ra polypeptide) can encode any appropriate polypeptide that can inhibit an IL-1 polypeptide. For example, a viral vector having a genome (e.g., a single-stranded or double-stranded DNA genome) including a nucleic acid sequence (e.g., an engineered DNA sequence) including (a) a nucleotide sequence encoding a BMP polypeptide and/or (b) a nucleotide sequence encoding a polypeptide that can inhibit an IL-1 polypeptide can include nucleic acid encoding any appropriate polypeptide that can inhibit an IL-1 polypeptide. Examples of polypeptides that can inhibit an IL-1 polypeptide include, without limitation, IL-1Ra polypeptides and IL-1R polypeptides (e.g., soluble IL-1R polypeptides). In some cases, a polypeptide that can inhibit an IL-1 polypeptide that can be encoded by a nucleic acid encoding a polypeptide that can inhibit an IL-1 polypeptide in a vector provided herein can have the amino acid sequence set forth in the NCBI databases at Accession No. NM_173842.3. In some cases, a polypeptide that can inhibit an IL-1 polypeptide that can be encoded by a nucleic acid encoding a polypeptide that can inhibit an IL-1 polypeptide in a vector provided herein can have the amino acid sequence set forth in SEQ ID NO:3. In some cases, a polypeptide that can inhibit an IL-1 polypeptide that can be encoded by a nucleic acid encoding a polypeptide that can inhibit an IL-1 polypeptide in a vector provided herein can comprise, consist essentially of, or consist of the amino acid sequence set forth in SEQ ID NO:3. In some cases, a polypeptide that can inhibit an IL-1 polypeptide that can be encoded by a nucleic acid encoding a polypeptide that can inhibit an IL-1 polypeptide in a vector provided herein can comprise, consist essentially of, or consist of the amino acid sequence set forth in SEQ ID NO:1 with one, two, three, four, five, six, seven, eight, nine, or ten amino acid deletions, additions, substitutions, or combinations thereof. In some cases, a BMP polypeptide that can be encoded by a nucleic acid encoding a BMP polypeptide in a vector provided herein can comprise, consist essentially of, or consist of the amino acid sequence set forth in SEQ ID NO:1 with two or less, three or less, four or less, five or less, six or less, seven or less, eight or less, nine or less, or ten or less amino acid deletions, additions, substitutions, or combinations thereof.

In some cases, a nucleic acid sequence including (a) a nucleotide sequence encoding a BMP polypeptide (e.g., a BMP2 polypeptide) and/or (b) a nucleotide sequence encoding a polypeptide that can inhibit an IL-1 polypeptide (e.g., an IL-1Ra polypeptide) can include one or more regulatory elements operably linked to the nucleotide sequence encoding a BMP polypeptide and/or to the nucleotide sequence encoding a polypeptide that can inhibit an IL-1 polypeptide. For example, a vector having a genome (e.g., a single-stranded or double-stranded DNA genome) including a nucleic acid sequence (e.g., an engineered DNA sequence) including (a) a nucleotide sequence encoding a BMP polypeptide and/or (b) a nucleotide sequence encoding a polypeptide that can inhibit an IL-1 polypeptide can include one or more regulatory elements operably linked to the nucleotide sequence encoding a BMP polypeptide. For example, a viral vector having a genome (e.g., a single-stranded or double-stranded DNA genome) including a nucleic acid sequence (e.g., an engineered DNA sequence) including (a) a nucleotide sequence encoding a BMP polypeptide and/or (b) a nucleotide sequence encoding a polypeptide that can inhibit an IL-1 polypeptide can include one or more regulatory elements operably linked to the nucleotide sequence encoding a polypeptide that can inhibit an IL-1 polypeptide. Such regulatory elements can include, without limitation, promoter sequences, enhancer sequences, response elements, signal peptides, translation initiation sites (e.g., Kozak sequences), internal ribosome entry sequences, polyadenylation signals, terminators, and inducible elements that modulate expression (e.g., transcription or translation) of a nucleic acid. The choice of regulatory element(s) that can be included in a vector (e.g., a viral vector or a non-viral vector) provided herein (e.g., a viral vector designed to express (a) a nucleotide sequence encoding a BMP polypeptide and/or (b) a nucleotide sequence encoding a polypeptide that can inhibit an IL-1 polypeptide) can depend on several factors, including, without limitation, inducibility, targeting, and the level of expression desired. For example, a promoter can be included in a vector provided herein to facilitate transcription of a nucleic acid encoding a BMP polypeptide, and/or a nucleotide sequence encoding a polypeptide that can inhibit an IL-1 polypeptide. A promoter can be a naturally occurring promoter or a recombinant promoter. A promoter can be constitutive or inducible (e.g., in the presence of tetracycline), and can affect the expression of a nucleic acid encoding a polypeptide in a general or tissue-specific manner.

The term “operably linked” as used herein with respect to a regulatory element and a nucleotide sequence encoding a BMP polypeptide (e.g., a BMP2 polypeptide) and/or a nucleotide sequence encoding a polypeptide that can inhibit an IL-1 polypeptide (e.g., an IL-1Ra polypeptide) refers to positioning of the regulatory element relative to the nucleic acid encoding a nucleotide sequence encoding a BMP polypeptide and/or a nucleotide sequence encoding a polypeptide that can inhibit an IL-1 polypeptide in such a way as to permit or facilitate expression of the BMP polypeptide and/or the IL-1Ra polypeptide. For example, a vector (e.g., a viral vector or a non-viral vector) provided herein (e.g., a viral vector designed to express (a) a nucleotide sequence encoding a BMP polypeptide and/or (b) a nucleotide sequence encoding a polypeptide that can inhibit an IL-1 polypeptide) can contain a promoter and nucleic acid encoding a BMP polypeptide. In this case, the promoter can be operably linked to the nucleic acid encoding a BMP polypeptide such that it drives expression of the BMP polypeptide in cells. Examples of promoters that can be used to drive expression of (a) a nucleotide sequence encoding a BMP polypeptide and/or (b) a nucleotide sequence encoding a polypeptide that can inhibit an IL-1 polypeptide include, without limitation, CMV promoters, EF1-α promoters, SV40 promoters, Cbh promoters, T7 promoters, RSV promoters, PGK promoters, CAG promoters, and β-actin promoters.

In some cases, a nucleotide sequence encoding a BMP polypeptide (e.g., a BMP2 polypeptide) and a nucleic acid sequence encoding a polypeptide that can inhibit an IL-1 polypeptide (e.g., an IL-1Ra polypeptide) can be present on separate vectors. For example, a first vector (e.g., a viral vector or a non-viral vector) can have a genome (e.g., a single-stranded or double-stranded DNA genome) including a nucleic acid sequence (e.g., an engineered DNA sequence) including a nucleotide sequence encoding a BMP polypeptide that is operably linked to a promoter, and a second vector (e.g., a viral vector or a non-viral vector) can have a genome (e.g., a single-stranded or double-stranded DNA genome) including a nucleic acid sequence (e.g., an engineered DNA sequence) including a nucleic acid sequence encoding a polypeptide that can inhibit an IL-1 polypeptide that is operably linked to a promoter.

In some cases, a nucleotide sequence encoding a BMP polypeptide (e.g., a BMP2 polypeptide) and a nucleic acid sequence encoding a polypeptide that can inhibit an IL-1 polypeptide (e.g., an IL-1Ra polypeptide) can be present on the same vector. For example, a single viral vector can have a genome (e.g., a single-stranded or double-stranded DNA genome) including a nucleic acid sequence (e.g., an engineered DNA sequence) including a promoter operably linked to a nucleotide sequence encoding a BMP polypeptide and a nucleic acid sequence encoding a polypeptide that can inhibit an IL-1 polypeptide where the nucleotide sequence encoding the BMP polypeptide and the nucleic acid sequence encoding the IL-1Ra polypeptide are separated by one or more features that allow for translation of two or more polypeptides from a single nucleic acid sequence (e.g., from a single messenger RNA (mRNA) transcript). Examples of features that allowing for translation of two or more polypeptides from a single nucleic acid sequence (e.g., from a single mRNA transcript) include, without limitation, internal ribosome entry sites (IRESs), nucleic acid sequences encoding a 2A polypeptide (e.g., P2A polypeptides, T2A polypeptides, E2A polypeptides, and F2A polypeptides), and polypeptide cleavage sites (e.g., furin cleavage sites). In some cases, a single viral vector can have a genome (e.g., a single-stranded or double-stranded DNA genome) that includes a nucleic acid sequence (e.g., an engineered DNA sequence) including a promoter operably linked to a nucleotide sequence encoding a BMP polypeptide and a nucleic acid sequence encoding a polypeptide that can inhibit an IL-1 polypeptide where the nucleotide sequence encoding the BMP polypeptide and the nucleic acid sequence encoding the IL-1Ra polypeptide are separated by a furin cleavage site.

In some cases, a single vector (e.g., a viral vector or a non-viral vector) provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be formulated into a composition (e.g., a pharmaceutically acceptable composition). For example, a single vector provided herein (e.g., a population of a single viral vector including (a) a nucleotide sequence encoding a BMP polypeptide and (b) a nucleotide sequence encoding a polypeptide that can inhibit an IL-1 polypeptide such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, collagen, polymers (e.g., polyvinylpyrrolidone (PVP), polyethylene glycol (PEG), poloxamer, crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), hyaluronic acid, glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, a single vector provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be formulated for oral or parenteral (including, without limitation, direct injection in situ, intravenous injection, intra-arterial injection, or intramuscular injection) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

Also provided herein are nucleic acid molecules (e.g., isolated nucleic acid molecules) including a nucleic acid sequence (e.g., an engineered DNA sequence) including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide).

Also provided herein are cells (e.g., host cells) containing one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide). In some cases, a host cell containing one or more vectors provided herein can be an in vitro cell (e.g., a cell in an in vitro culture).

Also provided herein are cells (e.g., host cells) containing a nucleic acid sequence (e.g., an engineered DNA sequence) including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide). In some cases, a host cell containing a nucleic acid sequence (e.g., an engineered DNA sequence) including (a) a nucleotide sequence encoding a BMP polypeptide and (b) a nucleotide sequence encoding a polypeptide that can inhibit an IL-1 polypeptide can be an in vitro cell (e.g., a cell in an in vitro culture).

Examples of cells (e.g., host cells) that can contain one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) and/or a nucleic acid sequence (e.g., an engineered DNA sequence) including (a) a nucleotide sequence encoding a BMP polypeptide and (b) a nucleotide sequence encoding a polypeptide that can inhibit an IL-1 polypeptide include, without limitation, fibroblasts, mesenchymal stromal cells, chondrocytes, induced pluripotent stem cells (iPSCs), HEK 293 cells, macrophages, immune cells, and myoblasts.

Also provided herein are methods for using a composition (e.g., a pharmaceutically acceptable composition) including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide). In some cases, a composition including one or more vectors provided herein can be administered to a mammal (e.g., a human) having a disease, disorder, or condition associated with bone loss to promote bone growth and/or bone regeneration within the mammal. For example, a composition including one or more vectors provided herein can be used to increase expression of a BMP polypeptide and a polypeptide that can inhibit an IL-1 polypeptide by cells within a mammal (e.g., a human) having a disease, disorder, or condition associated with bone loss to promote bone growth and/or bone regeneration within the mammal.

Any appropriate mammal having a disease, disorder, or condition associated with bone loss can be treated as described herein (e.g., by administering one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide)). Examples of mammals that can have a disease, disorder, or condition associated with bone loss and that can be treated as described herein include, without limitation, humans, non-human primates (e.g., monkeys), dogs, cats, horses, cows, pigs, sheep, mice, rats, rabbits, and hamsters. In some cases, a human having a disease, disorder, or condition associated with bone loss can be treated as described herein.

A mammal (e.g., a human) having any type of disease, disorder, or condition associated with bone loss can be treated as described herein (e.g., by administering a one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide)). Examples of diseases, disorders, and conditions associated with bone loss that can be treated as described herein include, without limitation, bone fractures (e.g., skeletal bone fractures), osteoporosis, aseptic loosening of prosthetic joints, maxillofacial applications, spine fusion, segmental bone defects, osteochondral defects, avascular necrosis or infarction of bone, osteoarthritis (e.g., erosive osteoarthritis (EOA)), rheumatoid arthritis, metastatic bone disease, bone grafting procedures, and cartilage grafting procedures.

In some cases, a composition including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal in need thereof (e.g., a human having a disease, disorder, or condition associated with bone loss) to increase expression of a BMP polypeptide and a polypeptide that can inhibit an IL-1 polypeptide by cells within the mammal. In some cases, the materials and methods described herein can be used to increase expression of a BMP polypeptide and a polypeptide that can inhibit an IL-1 polypeptide by cells within a mammal having a disease, disorder, or condition associated with bone loss by, for example, 10, 20, 30, 40, 50, 60, 70, 80, 90, 95, or more percent.

A composition including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can increase expression of a BMP polypeptide and a polypeptide that can inhibit an IL-1 polypeptide in any appropriate type of cells within a mammal (e.g., a human). In some cases, a composition including one or more vectors provided herein can increase expression of a BMP polypeptide and a polypeptide that can inhibit an IL-1 polypeptide in bone cells, cartilage cells, fibroblasts, muscle cells, immune cells, periosteal cells, osteoprogenitor cells, macrophages, neutrophils, and lymphocytes.

In some cases, a composition including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal in need thereof (e.g., a human having a disease, disorder, or condition associated with bone loss) to increase bone growth and/or bone regeneration within the mammal. In some cases, the materials and methods described herein can be used to increase bone growth and/or bone regeneration within a mammal having a disease, disorder, or condition associated with bone loss by, for example, 10, 20, 30, 40, 50, 60, 70, 80, 90, 95, or more percent.

In some cases, a composition including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal in need thereof (e.g., a human having a disease, disorder, or condition associated with bone loss) to reduce bone loss within the mammal. In some cases, the materials and methods described herein can be used to reduce bone loss within a mammal having a disease, disorder, or condition associated with bone loss by, for example, 10, 20, 30, 40, 50, 60, 70, 80, 90, 95, or more percent.

In some cases, the methods described herein can include identifying a mammal (e.g., a human) as having a disease, disorder, or condition associated with bone loss. Any appropriate method can be used to identify a mammal as having a disease, disorder, or condition associated with bone loss. For example, physical examinations, imaging techniques (e.g., x-rays, bone scans, magnetic resonance imaging, and CAT scans), clinical history, tissue biopsy, and serum/urine laboratory studies can be used to identify a mammal as having a disease, disorder, or condition associated with bone loss.

Any appropriate method can be used to deliver a composition (e.g., a pharmaceutically acceptable composition) including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) to a mammal (e.g., a human) having a disease, disorder, or condition associated with bone loss. In some cases, a composition including one or more vectors provided herein can be administered locally or systemically. In some cases, a composition including one or more vectors provided herein can be administered locally via direct injection in situ, intravenous injection, intra-arterial injection, or intramuscular injection. In some cases, a composition including one or more vectors provided herein can be administered via a carrier or scaffold such as a collagen sponge (e.g., a bovine collagen sponge carrier.

Any appropriate amount (e.g., any appropriate dose) of a composition (e.g., a pharmaceutically acceptable composition) including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal (e.g., a human) having a disease, disorder, or condition associated with bone loss. An effective amount of a composition including one or more vectors provided herein can be any amount that can treat a mammal having a disease, disorder, or condition associated with bone loss as described herein (e.g., that can increase expression of a BMP polypeptide by cells within the mammal) without producing significant toxicity to the mammal. In some cases, an effective amount of a composition including one or more vectors provided herein can be from about 1×10⁸ viral genomes to about 1×10¹³ viral genomes. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the disease, disorder, or condition associated with bone loss in the mammal being treated may require an increase or decrease in the actual effective amount administered.

A composition (e.g., a pharmaceutically acceptable composition) including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal (e.g., a human) having a disease, disorder, or condition associated with bone loss in any appropriate frequency. The frequency of administration can be any frequency that can treat a mammal having a disease, disorder, or condition associated with bone loss as described herein (e.g., that can increase expression of a BMP polypeptide by cells within the mammal) without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition (e.g., a pharmaceutically acceptable composition) including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) can be administered to a mammal (e.g., a human) having a disease, disorder, or condition associated with bone loss for any appropriate duration. An effective duration for administering or using a composition including one or more vectors provided herein can be any duration that can treat a mammal having a disease, disorder, or condition associated with bone loss as described herein (e.g., that can increase expression of a BMP polypeptide by cells within the mammal) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having a disease, disorder, or condition associated with bone loss can include administering to the mammal a composition (e.g., a pharmaceutically acceptable composition) including one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide) as the sole active ingredient to treat the mammal. For example, a composition including one or more vectors provided herein can include a population of a single viral vector that includes (a) a nucleotide sequence encoding a BMP polypeptide and (b) a nucleotide sequence encoding a polypeptide that can inhibit an IL-1 polypeptide (e.g., a population of viral vectors including a promotor sequence operably linked to a nucleic acid comprising (a) a nucleotide sequence encoding a BMP polypeptide and (b) a nucleotide sequence encoding a polypeptide that can inhibit an IL-1 polypeptide) as the sole active ingredient in the composition that is effective to treat a mammal having a disease, disorder, or condition associated with bone loss.

In some cases, methods for treating a mammal (e.g., a human) having a disease, disorder, or condition associated with bone loss as described herein (e.g., by administering one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide)) also can include administering to the mammal one or more (e.g., one, two, three, four, five or more) additional agents that can treat one or more symptoms of a disease, disorder, or condition associated with bone loss. Examples of additional agents that can be used to treat one or more symptoms of a disease, disorder, or condition associated with bone loss and can be administered together with a composition including one or more vectors provided herein include, without limitation, alendronate (e.g., BINOSTO® and FOSAMAX®), risedronate (e.g., ACTONEL® and ATELVIA®), ibandronate (e.g., BONIVA®), zoledronic acid (e.g., RECLAST® and ZOMETA®), denosumab (e.g., PROLIA® and XGEVA®), estrogen, raloxifene (e.g., EVISTA®), teriparatide (e.g., FORTEO®), abaloparatide (e.g., TYMLOS®), romosozumab (EVENITY®), and demineralized bone matrices (DBMs) (e.g., ALLOFUSE®, OSTEOSELECT®, OSTEOSPONGE®, H-GENIN, INTERGRO® DBM, ACCELL CONNEXUS®, ACCELL EVO3®, ACCELL TBM®, DYNAGRAFT® II, ORTHOBLAST® II, OPTIUM® DBM, PROGENIX® DBM Putty, DBX®, GRAFTON™, BIOSET® DBM, ALLOMATRIX™, and PUROS® DBM), and any combinations thereof. In cases where a composition including one or more vectors provided herein is used in combination with one or more additional agents used to treat one or more symptoms of a disease, disorder, or condition associated with bone loss, the one or more agents used to treat one or more symptoms of a disease, disorder, or condition associated with bone loss can be administered at the same time (e.g., in a single composition containing both the one or more vectors provided herein and the one or more agents used to treat one or more symptoms of a disease, disorder, or condition associated with bone loss) or independently. For example, a composition including one or more vectors provided herein can be administered first, and the one or more additional agents used to treat one or more symptoms of a disease, disorder, or condition associated with bone loss administered second, or vice versa.

In some cases, methods for treating a mammal (e.g., a human) having a disease, disorder, or condition associated with bone loss as described herein (e.g., by administering one or more vectors provided herein (e.g., a population of a single viral vector including (a) a nucleotide sequence encoding a BMP polypeptide (e.g., a BMP2 polypeptide) and (b) a nucleotide sequence encoding a polypeptide that can inhibit an IL-1 polypeptide (e.g., an IL-1Ra polypeptide) such as a viral vector including a promotor sequence operably linked to a nucleic acid comprising (a) a nucleotide sequence encoding a BMP polypeptide and (b) a nucleotide sequence encoding a polypeptide that can inhibit an IL-1 polypeptide)) also can include subjecting the mammal to one or more (e.g., one, two, three, four, five or more) additional therapies used to treat a disease, disorder, or condition associated with bone loss. Examples of additional therapies that can be used to treat a disease, disorder, or condition associated with bone loss include, without limitation, physical therapy, surgical implantation of a bone graft (e.g., an InFUSE® bone graft), bracing, casting, surgical implantation of hardware, and combinations thereof. In cases where a composition (e.g., a pharmaceutically acceptable composition) including one or more vectors provided herein is used in combination with one or more additional therapies used to treat a disease, disorder, or condition associated with bone loss, the one or more additional therapies can be performed at the same time or independently of the administration of a composition including one or more vectors provided herein. For example, one or more vectors provided herein can be administered before, during, or after the one or more additional therapies are performed.

The invention will be further described in the following examples, which do not limit the scope of the invention described in the claims.

### EXAMPLES

### Example 1: Exemplary Vectors

This Example describes the development of viral vectors that encode BMP-2 and IL-1Ra.

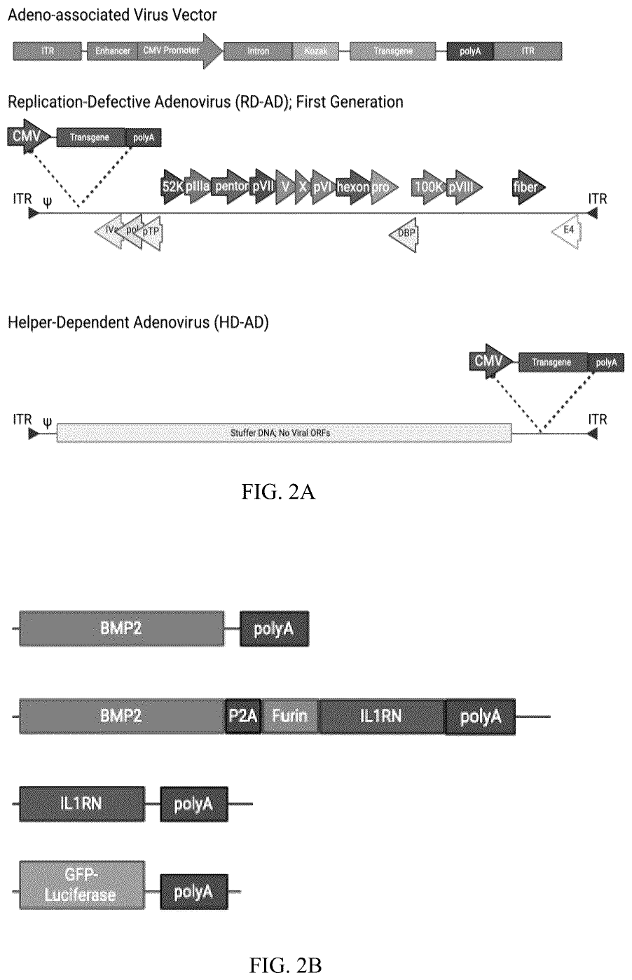

The genomic structures of the Ad vectors and AAV vectors are shown in FIG. 2A. A CMV promoter was used to drive transgene expression in all the vectors. A furin cleavage site between the BMP2 and IL-1Ra coding regions was used to allow the expression of both a BMP2 polypeptide and an IL-1Ra polypeptide from the vectors. Table 1 shows exemplary vectors constructed using the first-generation, replication defective Ad virus, AAV virus, and HD-Ad virus.

TABLE 1
Exemplary vector constructs.

| Backbone | Construct Name | Vector Contents |
|---|---|---|
| AAV | AAV.GL | nucleotide sequence encoding a green fluorescent |
| | | protein (GFP)-luciferase fusion protein (GL) |
| | AAV.IL-1Ra | nucleotide sequence encoding an IL-1Ra |
| | | polypeptide |
| | AAV.BMP-2 | nucleotide sequence encoding a BMP2 |
| | | polypeptide |
| | AAV.BMP2.IL-1Ra | nucleotide sequence encoding a BMP2 |
| | | polypeptide, a nucleotide sequence encoding a |
| | | P2A polypeptide, a furin cleavage site, and an IL- |
| | | 1Ra polypeptide |
| Replication | Ad.GL | nucleotide sequence encoding a GL polypeptide |
| defective | Ad.IL-1Ra | nucleotide sequence encoding an IL-1Ra |
| adenovirus | | polypeptide |
| | Ad.BMP-2 | nucleotide sequence encoding a BMP2 |
| | | polypeptide |
| | Ad.BMP-2.IL-1Ra | nucleotide sequence encoding a BMP2 |
| | | polypeptide, a nucleotide sequence encoding a |
| | | P2A polypeptide, a furin cleavage site, and an IL- |
| | | 1Ra polypeptide |
| helper- | HC-Ad.GL | nucleotide sequence encoding a GL polypeptide |
| dependent | HC-Ad.IL-1Ra | nucleotide sequence encoding an IL-1Ra |
| adenovirus | | polypeptide |
| | HC.Ad.BMP-2 | nucleotide sequence encoding a BMP2 |
| | | polypeptide |
| | HC.Ad.BMP-2.IL-1Ra | nucleotide sequence encoding a BMP2 |
| | | polypeptide and an IL-1Ra polypeptide with a |
| | | furin cleavage site separating the BMP2 and IL- |
| | | 1Ra coding regions |

Schematics of exemplary constructs are shown in FIG. 2B.

Replication-defective adenoviral vectors were amplified in HEK293 cells and purified by double cesium chloride banding. Single-cycle adenoviruses were rescued in 293-IIIA cells and purified by cesium chloride banding.

HD-adenovirus vectors were produced using the Cre/loxP system. The HD-adenovirus genome was a plasmid containing the adenoviral inverted terminal repeats (ITRs) required for vector genome replication, the y packaging sequence required for encapsidation, noncoding eukaryotic “stuffer” DNA to bring the vector genome size within the size range (27.7 kb to 37 kb) for efficient packaging into virions, and the expression cassette encoding BMP-2±IL-1Ra under the transcriptional control of a CMV promoter. The HD-adenoviral vector was designed to contain a furin-sensitive linker placed between the BMP-2 and IL-1Ra coding regions for expression of both a BMP2 polypeptide and an IL-1Ra polypeptide. To convert the plasmid form of the HD-adenovirus genome into the viral form, 293 cells expressing the site-specific Cre recombinase were transfected with the linearized HD-adenovirus genome. The transfected cells were subsequently infected with helper virus, an E1-deleted first-generation adenovirus bearing a packaging signal flanked by loxP sites to excise the packaging signal is excised from the helper virus genome by Cre-mediated site-specific recombination between the loxP sites. This rendered the helper virus genome unpackageable but still able to undergo DNA replication and thus complemented the replication and encapsidation of the HD-adenovirus genome. Vectors were purified by double cesium chloride banding. Virus particle concentration was determined by the optical density at 260 nm, and viral infectious unit was determined with an Adenovirus Rapid Titer kit (Invitrogen) on the 293 cells.

### Example 2: Vectors Encoding a BMP2 Polypeptide and an IL-1Ra Polypeptide Promote Bone Growth

In 2002, recombinant, human (rh) BMP-2 was approved by the United States Food and Drug Administration (FDA) for clinical use. Despite this, the realized clinical benefit of rhBMP2 has been marginal, and its approved indications remain limited to high-energy open tibial fractures, spinal fusions, and oromaxillofacial applications. Nevertheless, it is used in an off-label fashion to treat long bone non-unions (Ong et al., Spine (Phila Pa 1976), 35:1794-1800 (2010)). Because the clinical potency of rhBMP-2 is mediocre, it is used at very high, supraphysiological concentrations that not only raise costs, but also elicit side-effects, some of them severe, including heterotopic bone formation, radiculopathy, urogenital adverse events and wound complications. Ironically, the high doses of BMP-2 needed to form bone also induce osteoclastic activity. However, the most common side-effect of rhBMP2 is severe, dose-dependent inflammation, which can incur life-threatening sequelae (James et al., Tissue Eng. Part B Rev, 22:284-297 (2016); Carragee et al., Spine J, 11:471-491 (2011); and Shields et al., Spine (Phila Pa 1976), 31:542-547 (2006)).

This Example describes the discovery that expression of a BMP2 polypeptide and an IL-1Ra polypeptide encoded by a single viral vector can reduce bone loss or increase bone growth within a mammal (e.g., without inducing a significant adverse inflammatory immune response within the mammal).

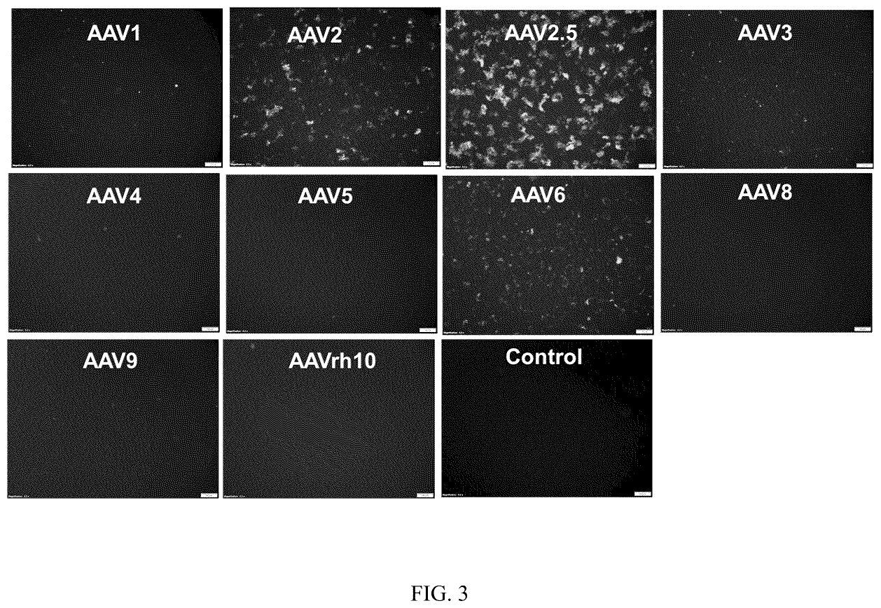

Monolayer cultures of rat MSCs were transduced with AAV of different serotypes encoding a GL fusion protein at a moi of 10,000. Transduction was assessed 48 hours later by fluorescent microscopy. AAV serotypes 2.5, 2 and, to a lesser degree, 6 transduced monolayer cultures of rat MSCs (FIG. 3).

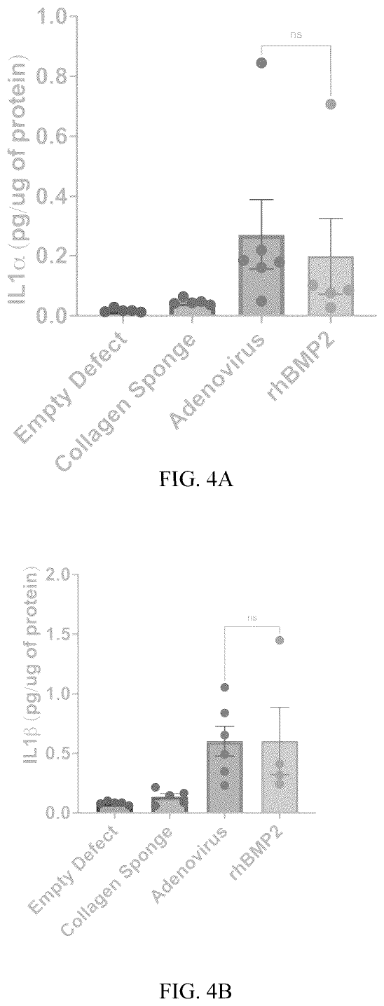

Critical size segmental defects were surgically created in the femora of rats. Into these defects were implanted a collagen sponge, a collagen sponge impregnated with 11 μg BMP-2, a replication defective adenovirus encoding BMP-2 or nothing at all (empty defect). Five days later, rats were euthanized and the amount of IL-la and IL-1B in the defects measured by ELISA. IL-1 levels remained low in the empty defects and those receiving the sponge alone (FIG. 4). IL-1 levels rose in response to adenovirus and BMP-2.

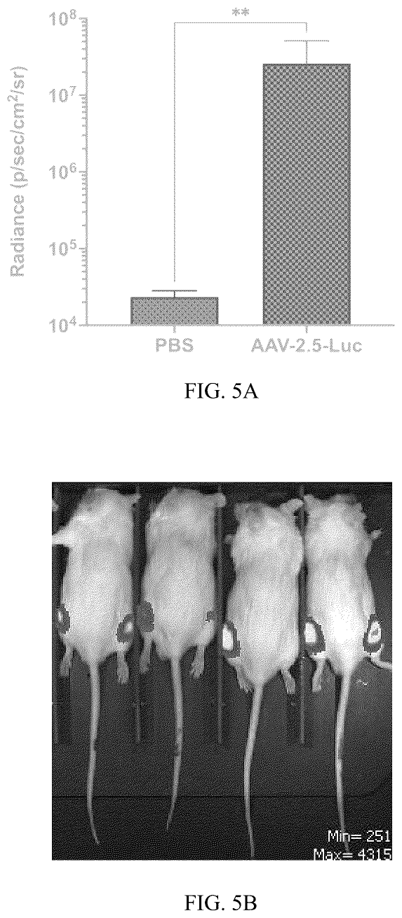

Critical size segmental defects were surgically created in the femora of rats. AAV2.5GL was implanted into these defects on a collagen sponge and luciferase activity visualized and measured by an in vivo imaging system (IVIS). At one week there was a robust expression of luciferase within the defects measured by luminometry (FIG. 5A) and imaging (FIG. 5B).

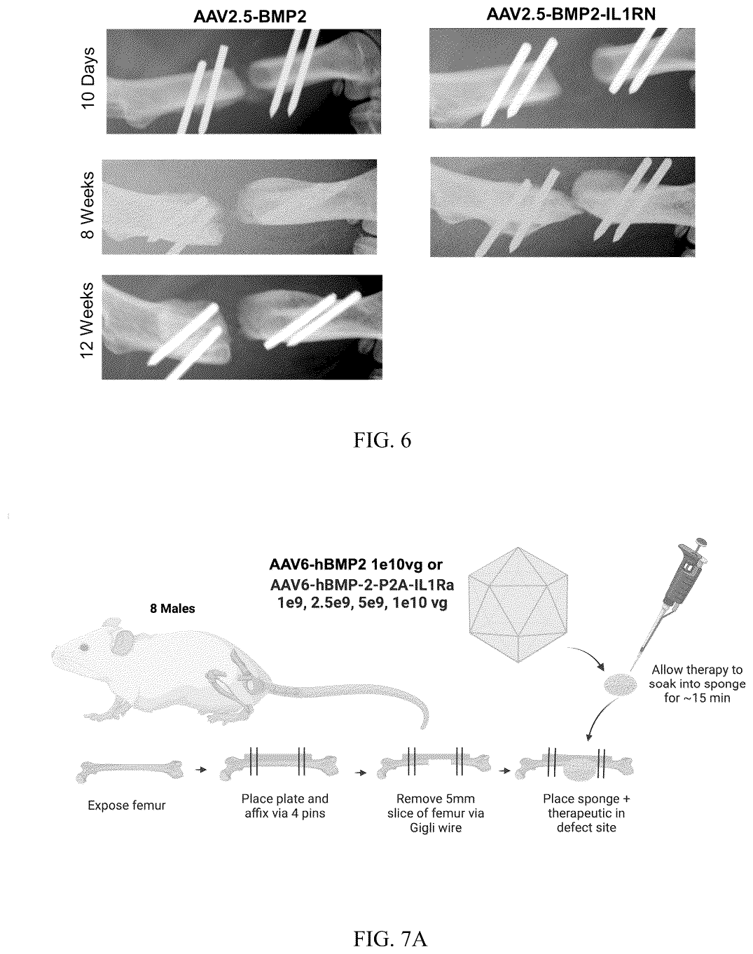

Critical size segmental defects were surgically created in the femora of rats. Defects received a very low dose (1×10⁸ viral genomes) of AAV2.5 encoding BMP2 alone (AAV2.5-BMP2) or both BMP2 and IL-1Ra (AVV2.5-BMP2-IL1RN). Healing of defects was monitored by periodic X-ray. At this dose, AAV2.5-BMP2 produced no healing of the defect whereas AAV2.5-BMP2-IL1RN induced significant new bone formation that at 8 weeks was close to bridging the defect (FIG. 6).

### Example 3: Exemplary BMP2 Sequences

Amino acid sequence of an exemplary BMP2
polypeptide
(SEQ ID NO: 1)
MVAGTRCLLALLLPQVLLGGAAGLVPELGRRKFAAASSGRPSSQP
SDEVLSEFELRLLSMFGLKQRPTPSRDAVVPPYMLDLYRRHSGQP
GSPAPDHRLERAASRANTVRSFHHEESLEELPETSGKTTRRFFFN
LSSIPTEEFITSAELQVFREQMQDALGNNSSFHHRINIYEIIKPA
TANSKFPVTRLLDTRLVNQNASRWESFDVTPAVMRWTAQGHANHG
FVVEVAHLEEKQGVSKRHVRISRSLHQDEHSWSQIRPLLVTFGHD
GKGHPLHKREKRQAKHKQRKRLKSSCKRHPLYVDFSDVGWNDWIV
APPGYHAFYCHGECPFPLADHLNSTNHAIVQTLVNSVNSKIPKAC
CVPTELSAISMLYLDENEKVVLKNYQDMVVEGCGCR
Nucleic acid sequence encoding SEQ ID NO: 1
(SEQ ID NO: 2)
ATGGTGGCCGGGACCCGCTGTCTTCTAGCGTTGCTGCTTCCCCAG
GTCCTCCTGGGCGGCGCGGCTGGCCTCGTTCCGGAGCTGGGCCGC
AGGAAGTTCGCGGCGGCGTCGTCGGGCCGCCCCTCATCCCAGCCC
TCTGACGAGGTCCTGAGCGAGTTCGAGTTGCGGCTGCTCAGCATG
TTCGGCCTGAAACAGAGACCCACCCCCAGCAGGGACGCCGTGGTG
CCCCCCTACATGCTAGACCTGTATCGCAGGCACTCAGGTCAGCCG
GGCTCACCCGCCCCAGACCACCGGTTGGAGAGGGCAGCCAGCCGA
GCCAACACTGTGCGCAGCTTCCACCATGAAGAATCTTTGGAAGAA
CTACCAGAAACGAGTGGGAAAACAACCCGGAGATTCTTCTTTAAT
TTAAGTTCTATCCCCACGGAGGAGTTTATCACCTCAGCAGAGCTT
CAGGTTTTCCGAGAACAGATGCAAGATGCTTTAGGAAACAATAGC
AGTTTCCATCACCGAATTAATATTTATGAAATCATAAAACCTGCA
ACAGCCAACTCGAAATTCCCCGTGACCAGACTTTTGGACACCAGG
TTGGTGAATCAGAATGCAAGCAGGTGGGAAAGTTTTGATGTCACC
CCCGCTGTGATGCGGTGGACTGCACAGGGACACGCCAACCATGGA
TTCGTGGTGGAAGTGGCCCACTTGGAGGAGAAACAAGGTGTCTCC
AAGAGACATGTTAGGATAAGCAGGTCTTTGCACCAAGATGAACAC
AGCTGGTCACAGATAAGGCCATTGCTAGTAACTTTTGGCCATGAT
GGAAAAGGGCATCCTCTCCACAAAAGAGAAAAACGTCAAGCCAAA
CACAAACAGCGGAAACGCCTTAAGTCCAGCTGTAAGAGACACCCT
TTGTACGTGGACTTCAGTGACGTGGGGTGGAATGACTGGATTGTG
GCTCCCCCGGGGTATCACGCCTTTTACTGCCACGGAGAATGCCCT
TTTCCTCTGGCTGATCATCTGAACTCCACTAATCATGCCATTGTT
CAGACGTTGGTCAACTCTGTTAACTCTAAGATTCCTAAGGCATGC
TGTGTCCCGACAGAACTCAGTGCTATCTCGATGCTGTACCTTGAC
GAGAATGAAAAGGTTGTATTAAAGAACTATCAGGACATGGTTGTG
GAGGGTTGTGGGTGTCGC

### Example 4: Exemplary IL-1Ra Sequences

Amino acid sequence of an exemplary IL-1Ra
polypeptide
(SEQ ID NO: 3)
MEICRGLRSHLITLLLFLFHSETICRPSGRKSSKMQAFRIWDVNQ
KTFYLRNNQLVAGYLQGPNVNLEEKIDVVPIEPHALFLGIHGGKM
CLSCVKSGDETRLQLEAVNITDLSENRKQDKRFAFIRSDSGPTTS
FESAACPGWFLCTAMEADQPVSLTNMPDEGVMVTKFYFQEDE
Nucleic acid sequence encoding SEQ ID NO: 3
(SEQ ID NO: 4)
AGTCCACTGCCTTGCTGCAGTCACAGAATGGAAATCTGCAGAGGC
CTCCGCAGTCACCTAATCACTCTCCTCCTCTTCCTGTTCCATTCA
GAGACGATCTGCCGACCCTCTGGGAGAAAATCCAGCAAGATGCAA
GCCTTCAGAATCTGGGATGTTAACCAGAAGACCTTCTATCTGAGG
AACAACCAACTAGTTGCTGGATACTTGCAAGGACCAAATGTCAAT
TTAGAAGAAAAGATAGATGTGGTACCCATTGAGCCTCATGCTCTG
TTCTTGGGAATCCATGGAGGGAAGATGTGCCTGTCCTGTGTCAAG
TCTGGTGATGAGACCAGACTCCAGCTGGAGGCAGTTAACATCACT
GACCTGAGCGAGAACAGAAAGCAGGACAAGCGCTTCGCCTTCATC
CGCTCAGACAGTGGCCCCACCACCAGTTTTGAGTCTGCCGCCTGC
CCCGGTTGGTTCCTCTGCACAGCGATGGAAGCTGACCAGCCCGTC
AGCCTCACCAATATGCCTGACGAAGGCGTCATGGTCACCAAATTC
TACTTCCAGGAGGACGAGTAGTACTGCCCAGGCCTGCCTGTTCCC
ATTCTTGCATGGCAAGGACTGCAGGGACTGCCAGTCCCCCTGCCC
CAGGGCTCCCGGCTATGGGGGCACTGAGGACCAGCCATTGAGGGG
TGGACCCTCAGAAGGCGTCACAACAACCTGGTCACAGGACTCTGC
CTCCTCTTCAACTGACCAGCCTCCATGCTGCCTCCAGAATGGTCT
TTCTAATGTGTGAATCAGAGCACAGCAGCCCCTGCACAAAGCCCT
TCCATGTCGCCTCTGCATTCAGGATCAAACCCCGACCACCTGCCC
AACCTGCTCTCCTCTTGCCACTGCCTCTTCCTCCCTCATTCCACC
TTCCCATGCCCTGGATCCATCAGGCCACTTGATGACCCCCAACCA
AGTGGCTCCCACACCCTGTTTTACAAAAAAGAAAAGACCAGTCCA
TGAGGGAGGTTTTTAAGGGTTTGTGGAAAATGAAAATTAGGATTT
CATGATTTTTTTTTTTCAGTCCCCGTGAAGGAGAGCCCTTCATTT
GGAGATTATGTTCTTTCGGGGAGAGGCTGAGGACTTAAAATATTC
CTGCATTTGTGAAATGATGGTGAAAGTAAGTGGTAGCTTTTCCCT
TCTTTTTCTTCTTTTTTTGTGATGTCCCAACTTGTAAAAATTAAA
AGTTATGGTACTATGTTAGCCCCATAATTTTTTTTTTCCTTTTAA
AACACTTCCATAATCTGGACTCCTCTGTCCAGGCACTGCTGCCCA
GCCTCCAAGCTCCATCTCCACTCCAGATTTTTTACAGCTGCCTGC
AGTACTTTACCTCCTATCAGAAGTTTCTCAGCTCCCAAGGCTCTG
AGCAAATGTGGCTCCTGGGGGTTCTTTCTTCCTCTGCTGAAGGAA
TAAATTGCTCCTTGACATTGTAGAGCTTCTGGCACTTGGAGACTT
GTATGAAAGATGGCTGTGCCTCTGCCTGTCTCCCCCACCGGGCTG
GGAGCTCTGCAGAGCAGGAAACATGACTCGTATATGTCTCAGGTC
CCTGCAGGGCCAAGCACCTAGCCTCGCTCTTGGCAGGTACTCAGC
GAATGAATGCTGTATATGTTGGGTGCAAAGTTCCCTACTTCCTGT
GACTTCAGCTCTGTTTTACAATAAAATCTTGAAAATGCC

### Example 5: AAV6-BMP2-IL1RN Promotes Bone Regeneration Via Endochondral Ossification

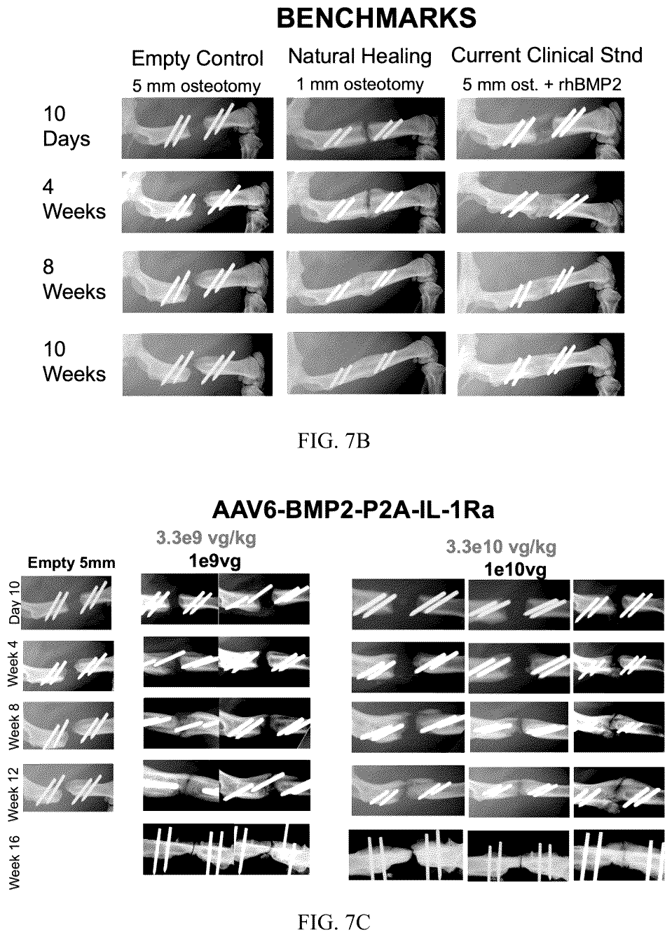

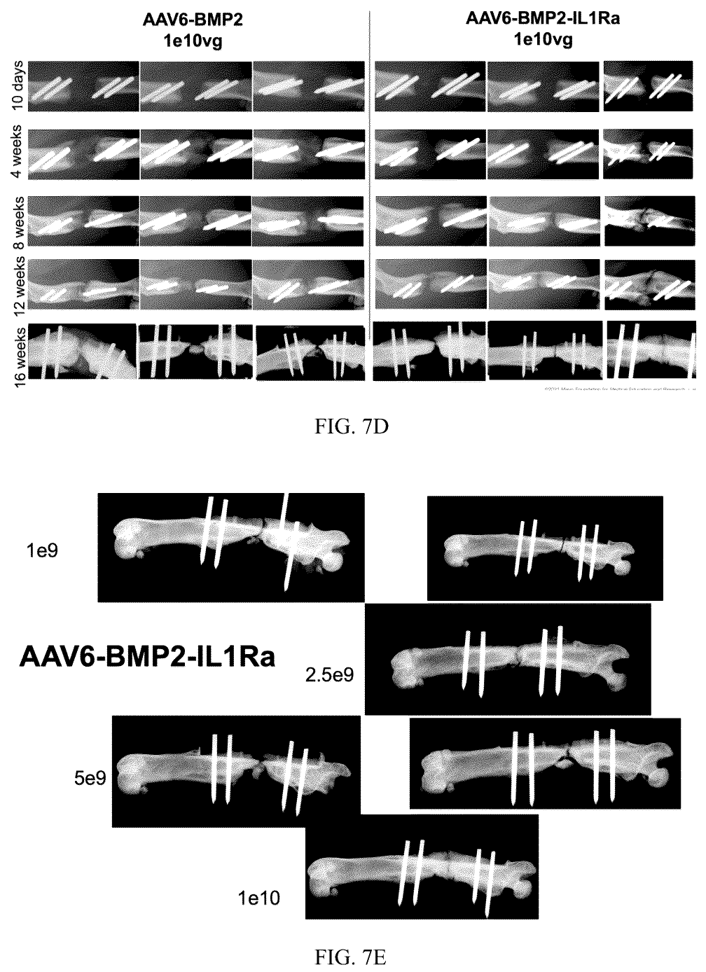

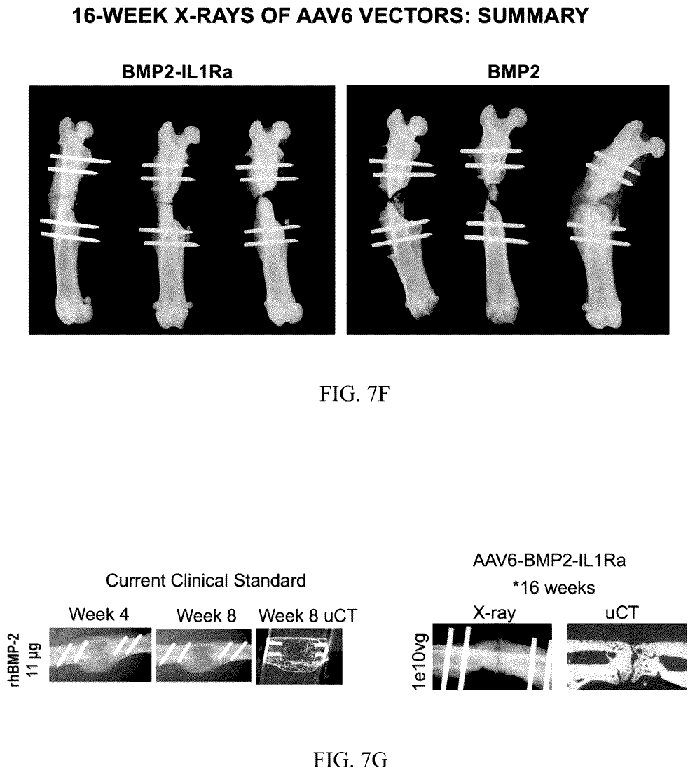

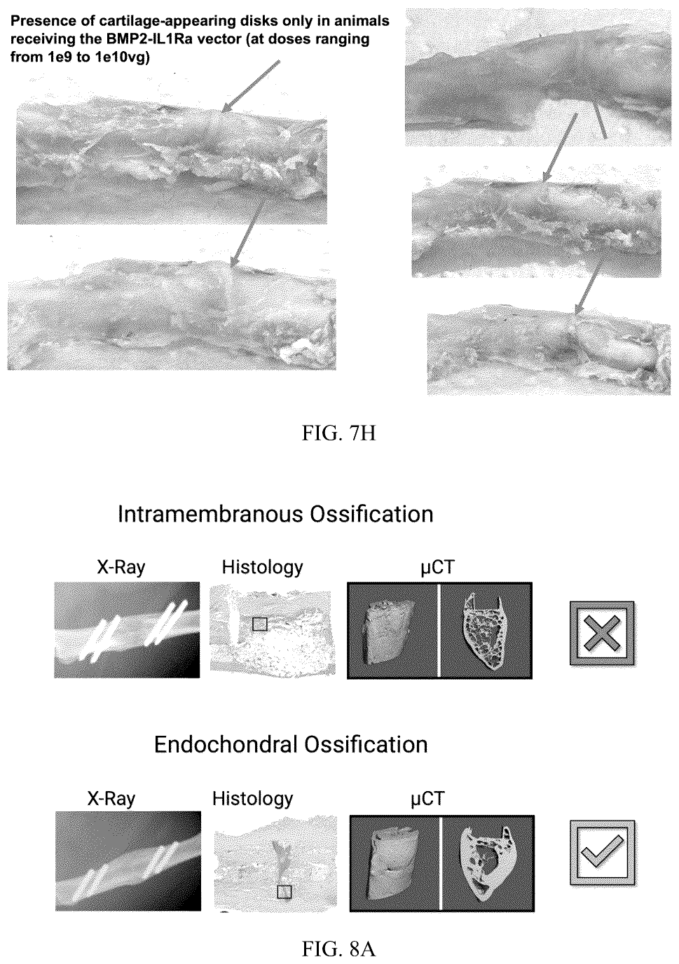

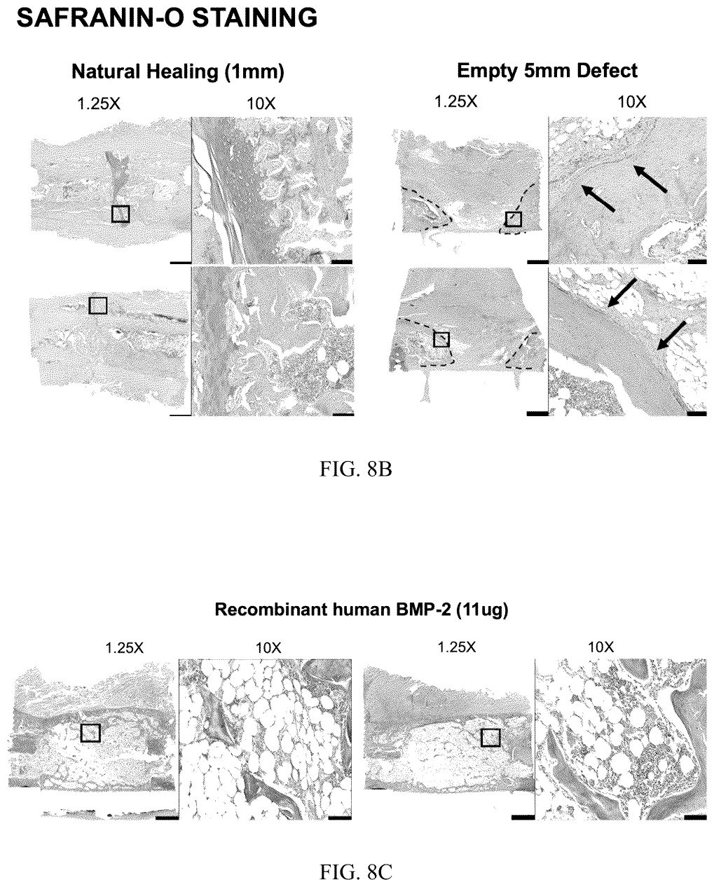

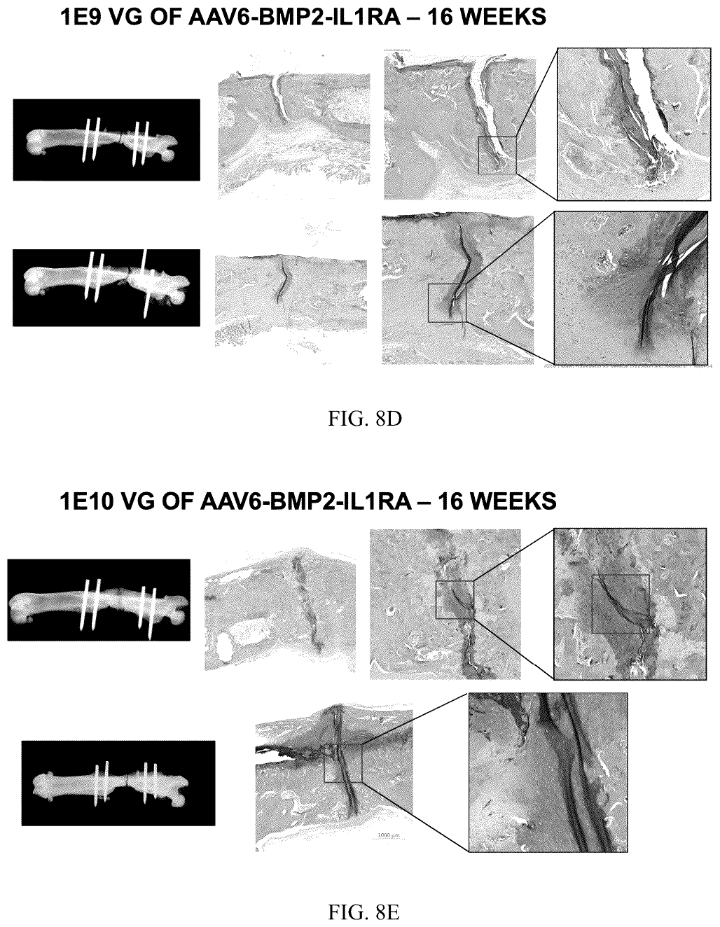

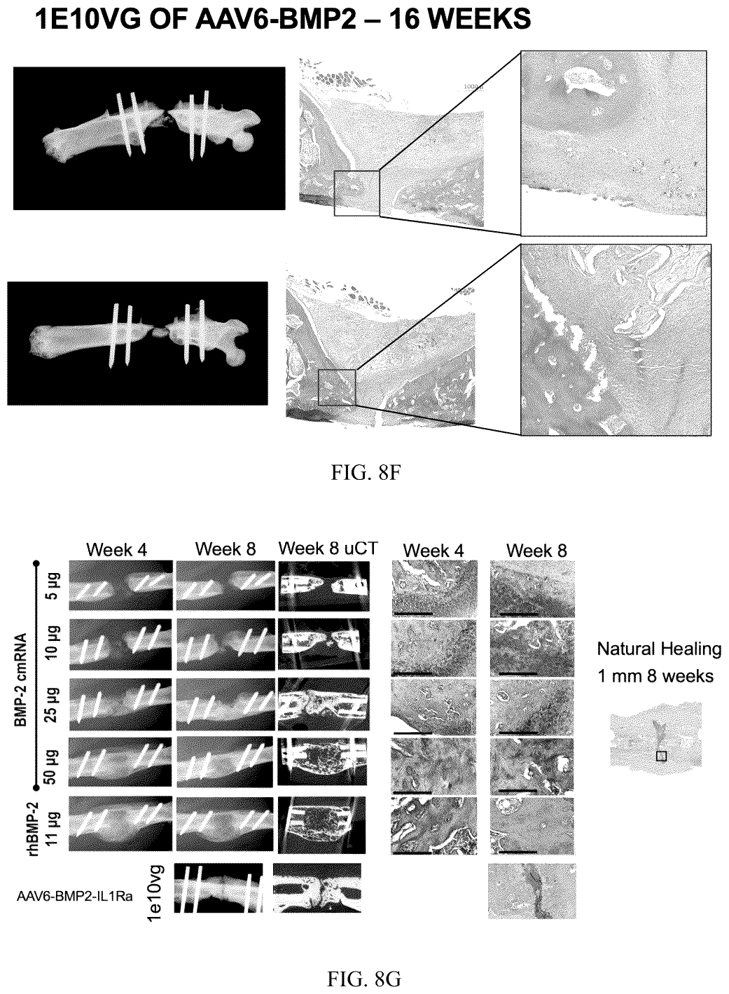

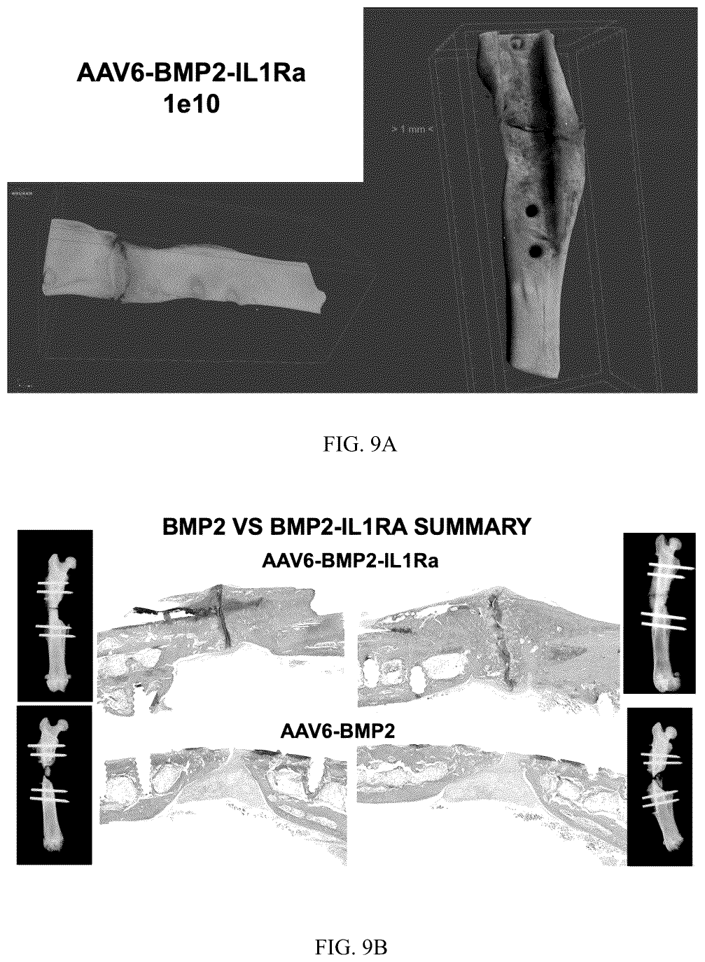

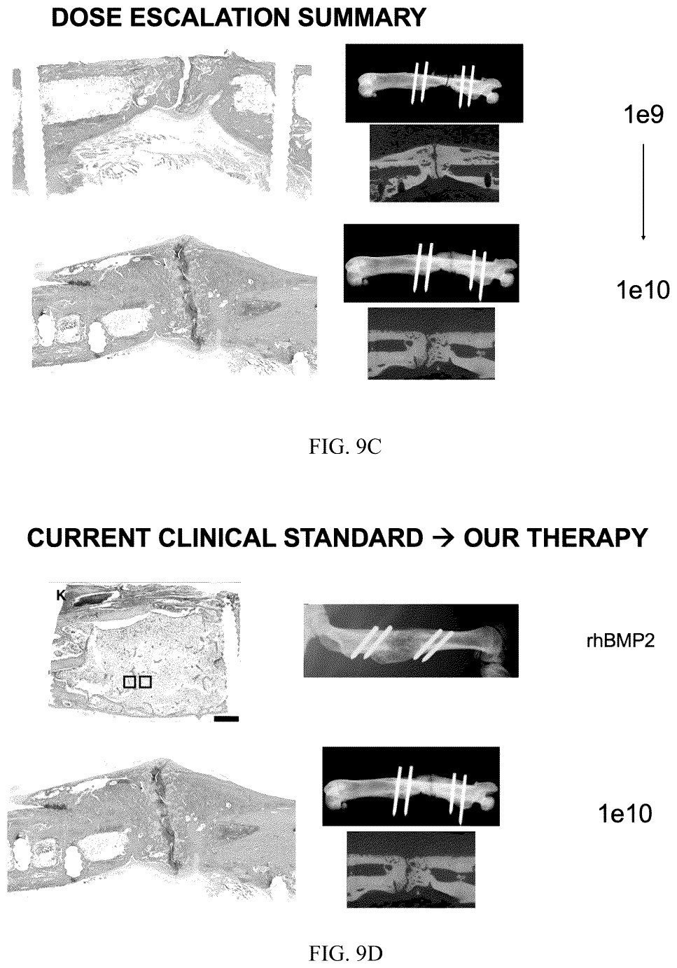

Critical size segmental defects were surgically created in rat femora. Into these defects, a collagen sponge containing rhBMP2 (11 μg), AAV6-BMP2 (1×10¹⁰ vg), or AAV6-BMP2-P2A-IL1Ra (1×10⁹ vg, 2.5×10⁹ vg, 5×10⁹ vg, and 1×10¹⁰ vg) was implanted. 1 mm segmental defects were surgically created with no implanted collagen sponge as a naturally healing control. Healing of defects was monitored by periodic X-ray imaging. 1×10¹⁰ vg AAV6-BMP2 promoted some healing of the defects whereas AAV6-BMP2-IL1RN induced significant new bone formation across all doses, and by week 16, it was close to bridging the defects via endochondral ossification (FIGS. 7A-7H, 8A-8G, and 9A-9D). At week 16, rats were euthanized and dissected. Femora were then imaged ex vivo using X-ray and micro-CT prior to proceeding with decalcification (10% EDTA for 28 days) for histology and safranin-O fast green staining (FIGS. 8A-8G and 9A-9D).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A vector comprising a promotor sequence operably linked to a nucleic acid comprising (a) a nucleotide sequence encoding a bone morphogenetic protein (BMP) polypeptide and (b) a nucleotide sequence encoding a polypeptide that can inhibit an interleukin-1 (IL-1) polypeptide.

2. The vector of claim 1, wherein said vector is a viral vector.

3. The vector of claim 2, wherein said viral vector is an adeno-associated viral vector or a helper-dependent adenoviral vector.

4. The vector of claim 1, wherein said promoter is selected from the group consisting of a CMV promoter, a EF1-α promoter, a SV40 promoter, a Cbh promoter, a T7 promoter, a RSV promoter, a PGK promoter, a CAG promoter, and a β-actin promoter.

5. The vector of claim 1, wherein said (a) and said (b) are separated by a nucleic acid sequence encoding a P2A polypeptide.

6. The vector of claim 1, wherein said (a) and said (b) are separated by a polypeptide cleavage site.

7. The vector of claim 6, wherein said polypeptide cleavage site is a furin cleavage site.

8. The vector of claim 1, wherein said (a) and said (b) are separated by an internal ribosome entry site.

9. The vector of claim 1, wherein said (a) and said (b) are separated by a second promoter.

10. The vector of claim 9, wherein said second promoter is selected from the group consisting of a CMV promoter, a EF1-α promoter, a SV40 promoter, a Cbh promoter, a T7 promoter, a RSV promoter, a PGK promoter, a CAG promoter, and a β-actin promoter.

11. The vector of claim 1, wherein said BMP polypeptide is a BMP2 polypeptide.

12. The vector of claim 1, wherein said BMP polypeptide comprises the amino acid sequence set forth in SEQ ID NO:1.

13. The vector of claim 1, wherein said polypeptide that can inhibit said IL-1 polypeptide is an IL-1Ra polypeptide.

14. The vector of claim 1, wherein said polypeptide that can inhibit said IL-1 polypeptide comprises the amino acid sequence set forth in SEQ ID NO:3.

15. A method for treating a mammal having a disease, disorder, or condition associated with bone loss, wherein said method comprises administering to said mammal a vector comprising a promotor sequence operably linked to a nucleic acid comprising (a) a nucleotide sequence encoding a bone morphogenetic protein (BMP) polypeptide and (b) a nucleotide sequence encoding a polypeptide that can inhibit an interleukin-1 (IL-1) polypeptide, wherein expression of said BMP polypeptide and said polypeptide that can inhibit said IL-1 polypeptide is increased within said mammal, and wherein bone loss is reduced within said mammal or bone growth is increased within said mammal.

16. The method of claim 15, wherein said mammal is a human.

17. The method of claim 15, wherein said disease, disorder, or condition is selected from the group consisting of a bone fracture, osteoporosis, aseptic loosening of prosthetic joints, maxillofacial applications, spine fusion, a segmental bone defect, an osteochondral defect, an avascular necrosis or infarction of bone, osteoarthritis, rheumatoid arthritis, a metastatic bone disease, a bone grafting procedure, and a cartilage grafting procedure.

18-20. (canceled)

21. A method for promoting bone growth within a mammal, wherein said method comprises administering to said mammal a vector comprising a promotor sequence operably linked to a nucleic acid comprising (a) a nucleotide sequence encoding a bone morphogenetic protein (BMP) polypeptide and (b) a nucleotide sequence encoding a polypeptide that can inhibit an interleukin-1 (IL-1) polypeptide, wherein expression of said BMP polypeptide and said polypeptide that can inhibit said IL-1 polypeptide is increased within said mammal, and wherein bone growth is increased within said mammal.

22-26. (canceled)

27. A composition comprising (1) a first vector comprising nucleic acid comprising a first promotor sequence operably linked to a nucleotide sequence encoding a bone morphogenetic protein (BMP) polypeptide and (2) a second vector comprising a second promotor sequence operably linked to a nucleotide sequence encoding a polypeptide that can inhibit an interleukin-1 (IL-1) polypeptide.

28. The composition of claim 27, wherein said first vector and said second vector are viral vectors.

29. The composition of claim 28, wherein said first vector and said second vector are independently an adeno-associated viral vector or a helper-dependent adenoviral vector.

30. The composition of claim 27, wherein said first promoter and said second promoter are independently selected from the group consisting of a CMV promoter, a EF1-α promoter, a SV40 promoter, a Cbh promoter, a T7 promoter, a RSV promoter, a PGK promoter, a CAG promoter, and a β-actin promoter.

31. (canceled)

32. The composition of claim 27, wherein said BMP polypeptide is a BMP2 polypeptide.

33. The composition of claim 27, wherein said BMP polypeptide comprises the amino acid sequence set forth in SEQ ID NO:1.

34. The composition of claim 27, wherein said polypeptide that can inhibit said IL-1 polypeptide is a IL-1Ra polypeptide.

35. The composition of claim 27, wherein said polypeptide that can inhibit said IL-1 polypeptide comprises the amino acid sequence set forth in SEQ ID NO:3.

36-47. (canceled)

48. A method for treating a mammal having a disease, disorder, or condition associated with bone loss, wherein said method comprises administering, to said mammal, (a) a first vector comprising nucleic acid comprising a first promotor sequence operably linked to a nucleotide sequence encoding a bone morphogenetic protein (BMP) polypeptide and (b) a second vector comprising a second promotor sequence operably linked to a nucleotide sequence encoding a polypeptide that can inhibit an interleukin-1 (IL-1) polypeptide, wherein expression of said BMP polypeptide and said polypeptide that can inhibit said IL-1 polypeptide is increased within said mammal, and wherein bone loss is reduced within said mammal or bone growth is increased within said mammal.

49-53. (canceled)

54. A method for promoting bone growth within a mammal, wherein said method comprises administering, to said mammal, (a) a first vector comprising nucleic acid comprising a first promotor sequence operably linked to a nucleotide sequence encoding a bone morphogenetic protein (BMP) polypeptide and (b) a second vector comprising a second promotor sequence operably linked to a nucleotide sequence encoding a polypeptide that can inhibit an interleukin-1 (IL-1) polypeptide, wherein expression of said BMP polypeptide and said polypeptide that can inhibit said IL-1 polypeptide is increased within said mammal, and wherein bone growth is increased within said mammal.

55-59. (canceled)


SEQUENCE LISTING (4 sequences)

SEQ ID NO: 1 - protein, 396 aa, synthetic construct
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - DNA, 1188 bp, synthetic construct
  source 1..1188: mol_type = other DNA
atggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgc

SEQ ID NO: 3 - protein, 177 aa, synthetic construct
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 4 - DNA, 1704 bp, synthetic construct
  source 1..1704: mol_type = other DNA
agtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcc